(19) United States
(12) Patent Application Publication
(10) Pub. No.: US 2020/0103396 A1
(43) Pub. Date: Apr. 2, 2020

(54) DEUTERIUM DEPLETION MEASUREMENT

(71) Applicant: T. Que COLLINS, Los Angeles, CA (US)

(72) Inventor: T. Que COLLINS, Los Angeles, CA (US)

(21) Appl. No.: 16/584,431

(22) Filed: Sep. 26, 2019

Related U.S. Application Data
(60) Provisional application No. 62/765,999, filed on Sep. 27, 2018.

(51) Int. Cl. G01N 33/50; G01N 33/52

(52) U.S. Cl. CPC G01N 33/5082; G01N 33/52

(57) ABSTRACT

Provided are methods of using non-invasive assays to determine deuterium levels in a subject to effect health, disease, and athletic performance.

### CROSS-REFERENCE

This application claims the benefit of U.S. Provisional Application No. 62/765,999 filed Sep. 27, 2018, which application is incorporated herein by reference.

### BACKGROUND

Deuterium, also referred to as deuteron, is a stable isotopic pair of hydrogen. Since deuterons may induce uncontrolled cellular proliferation and cause structural damage during molecule synthesis and energy transfer, protocols reducing the deuterium levels in a subject may provide a useful tool in treating diseases and disorder or improving overall health of the subject. As such, there is a need to non-invasively measure the deuterium levels in the subject and analyze the measurements in order to provide information about the health and condition of the subject.

### SUMMARY

Provided herein are methods to measure and lower deuterium levels in a subject. The subject may be administered one or more of various deuterium depletion protocols described herein designed to lower the deuterium level in the subject. Before, during, and after the deuterium depletion protocol, various biological samples are collected from the subject in a non-invasive or minimally invasive manner. The deuterium levels of the samples are measured to determine the deuterium levels at different time points. This can help track the progress of deuterium depletion, or the deuterium depletion rate, in the subject and their health conditions. The methods described herein provides various advantages, including the capability to non-invasively and repeatedly measuring deuterium levels in the subject and the ability to generate a more accurate indicator of the health and condition of the subject.

Provided herein are methods of measuring a deuterium level to determine a rate of deuterium depletion in a subject comprising: a) obtaining a first biological fluid from the subject; b) measuring an initial level of deuterium in the first biological fluid; c) providing a deuterium-depletion protocol to the subject; d) obtaining a subsequent biological fluid from the subject; and e) measuring a subsequent level of deuterium in the subsequent biological fluid, wherein the initial level and the subsequent level are used to determine a rate of deuterium depletion in the subject. In some embodiments, the deuterium-depletion protocol comprises providing a deuterium-depleted water to the subject. In some embodiments, the deuterium-depletion protocol comprises providing a deuterium-depleted nutritional diet to the subject. In some embodiments, the deuterium-depletion protocol comprises providing a ketogenic nutritional protocol to the subject. In some embodiments, the biological fluid comprises blood, serum, plasma, urine, saliva, tear, sweat, or breath condensate, stool, or spinal fluid. In some embodiments, the biological fluid comprises blood, serum, plasma, urine, saliva, or breath condensate. In some embodiments, the biological fluid is obtained from exhaled breath of the subject. In some embodiments, wherein steps d) and e) are repeated over a pre-determined period of time. In some embodiments, the pre-determined period of time is at least one week. In some embodiments, the deuterium depleted water comprises about 65 ppm to about 135 ppm deuterium. In some embodiments, the deuterium depleted water comprises about 65 ppm to about 125 ppm deuterium. In some embodiments, the deuterium-depleted nutritional diet comprises a natural plant derived diet, an animal fat derived diet, or a combination thereof. In some embodiments, the deuterium-depleted nutritional diet comprises a vegan diet. In some embodiments, the deuterium-depleted nutritional diet comprises plant or animal grown in low deuterium environment. In some embodiments, the deuterium-depleted nutritional diet comprises a diet having a predetermined deuterium content. In some embodiments, the deuterium-depletion protocol comprises an anti-cancer agent. In some embodiments, measuring of steps b) and e) comprises measuring deuterium level using isotope ratio mass spectrometry. In some embodiments, measuring of steps b) and e) comprises measuring deuterium level using water-based laser spectroscopy. In some embodiments, measuring of steps b) and e) comprises measuring a deuterium-proton ratio of the biological fluid. In some embodiments, the subject is a human. In some embodiments, the subject is an animal. In some embodiments, the subject is a plant. In some embodiments, the subject is a food item. In some embodiments, the measuring further comprises measuring of a tissue of interest in the individual using MM. In some embodiments, the biological fluid is breath condensate and blood. In some embodiments, the measuring comprises determining a weighted average of deuterium values from breath condensate and blood. In some embodiments, the deuterium-depletion protocol comprises providing a breathing protocol in an environment comprising water vapor having a deuterium level of no more than 135 ppm. In some embodiments, the deuterium-depletion protocol comprises exposure to red or near infra-red light for a pre-determined length of time. In some embodiments, the deuterium-depletion protocol comprises providing a wash protocol with a wash composition having a deuterium level of no more than 135 ppm. In some embodiments, the deuterium-depletion protocol comprises providing a topical composition having a deuterium level of no more than 135 ppm.

Provided herein are methods of measuring a deuterium level in a subject comprising: a) measuring an initial deuterium level in a tissue of interest the subject; b) providing a deuterium-depletion protocol to the subject; c) measuring a subsequent deuterium level in the tissue of interest in the subject, wherein the initial deuterium level and the subsequent deuterium level are used to determine a rate of deuterium depletion level in the subject, wherein the measuring of steps a) and c) comprises measuring deuterium level in the tissue of interest using MRI. In some embodiments, the deuterium-depletion protocol comprises providing a deuterium-depleted water to the subject. In some embodiments, the deuterium-depletion protocol providing a ketogenic nutritional protocol to the subject. In some embodiments, the measuring deuterium levels in tissues using MRI comprises measuring MRI tissue luminescence. In some embodiments, the measuring comprises proton (1H) nuclear MRI of the tissue of interest in the subject. In some embodiments, the measuring comprises determining a deuterium-proton ratio in the tissue of interest in the subject. In some embodiments, a decrease in the deuterium-proton ratio indicates deuterium depletion. In some embodiments, the tissue of interest comprises at least one of skin, muscle, and adipose tissue. In some embodiments, the subject is a human. In some embodiments, the subject is an animal. In some embodiments, the subject is a plant. In some embodiments, the subject is a food item. In some embodiments, the deuterium-proton ratio of the biological fluid below 1:5000 is indicative of deuterium depletion. In some embodiments, the measuring comprises using a 1.5 Tesla or lower magnetic field MM. In some embodiments, the measuring comprises using a T1 sequence. In some embodiments, the measuring comprises using a T1 sequence without contrast. In some embodiments, the measuring comprises measuring proton tunneling, wherein the proton tunneling measures free proton movements. In some embodiments, the deuterium-proton ratio in the tissue of interest in the subject below 1:5000 is indicative of deuterium depletion. In some embodiments, the measuring further comprises measuring a biological fluid obtained from the subject.

Provide herein are methods of treating a metabolic disease in a subject comprising: a) measuring an initial deuterium level in a first biological fluid from the subject; b) providing a deuterium-depletion protocol to the subject; c) measuring a subsequent deuterium level in a subsequent biological fluid, wherein the initial deuterium level and the subsequent deuterium level are used to determine a status of the metabolic disease in the subject. In some embodiments, the deuterium-depletion protocol comprises a diet comprising food having deuterium level of no more than 135 ppm. In some embodiments, the deuterium-depletion protocol comprises exposure to red and near-infra-red light. In some embodiments, the deuterium-depletion protocol comprises a breathing method to enhance deuterium depletion. In some embodiments, the deuterium-depletion protocol comprises a method to increase breath fractionation. In some embodiments, the deuterium-depletion protocol comprises breathing in an environment filled with vapors having a deuterium level of no more than 135 ppm. In some embodiments, the deuterium-depletion protocol comprises a sleeping method to enhance deuterium depletion. In some embodiments, the deuterium-depletion protocol comprises cold and hot thermotherapy or thermogenesis. In some embodiments, the deuterium-depletion protocol comprises sound therapy. In some embodiments, the deuterium-depletion protocol comprises at least one of acupuncture, chiropractic manipulation, and massage therapy. In some embodiments, the deuterium-depletion protocol comprises an ozone and hyperbaric oxygen therapy. In some embodiments, the deuterium-depletion protocol comprises injecting a composition having a deuterium level of no more than 135 ppm into a tissue or a blood vessel of the subject. In some embodiments, the deuterium-depletion protocol comprises applying a topical composition having a deuterium level of no more than 135 ppm on a skin of the subject. In some embodiments, the deuterium-depletion protocol comprises administering washes and cleanses with a composition having a deuterium level of no more than 135 ppm to at least one of ear, eyes, nose, colon, vagina or penis. In some embodiments, the deuterium-depletion protocol comprises cryotherapy. In some embodiments, the metabolic disease comprises at least one of diabetes, acid-base imbalance, metabolic brain diseases, calcium metabolic disorders, DNA repair-deficiency disorders, glucose metabolism disorders, hyperlactemia, iron metabolism disorders, lipid metabolism disorders, malabsorption syndromes, metabolic syndrome X, inborn error of metabolism, enzyme deficiencies, mitochondrial diseases, phosphorus metabolism disorders, porphyrias, proteostasis deficiencies, metabolic skin diseases, wasting syndrome, and water-electrolyte imbalance. In some embodiments, the metabolic disease is diabetes. In some embodiments, the metabolic disease is cancer. In some embodiments, the cancer comprises a tumor of at least one of breast, heart, lung, small intestine, colon, spleen, kidney, bladder, head, neck, ovarian, prostate, brain, pancreatic, skin, bone, bone marrow, blood, thymus, uterine, testicular, liver, or combinations thereof. In some embodiments, the method results in an increase in ATP production. In some embodiments, the method results in an increase in metabolic water production. In some embodiments, the method results in improved cellular function. In some embodiments, the method mitigates the progression of the metabolic disease. In some embodiments, the method prevents growth of or reduces a tumor. In some embodiments, the method enhances creation of a biologically active molecule having a favorable three-dimensional structure. In some embodiments, the biologically active molecule comprises cholesterol, estrogen, or DNA. In some embodiments, the measuring comprises using water-based laser spectroscopy, isotope ratio mass spectrometry, or proton (1H) nuclear MRI. In some embodiments, the diet comprising food having deuterium level of no more than 135 ppm comprises at least one of vegan, vegetarian, ketogenic, or low carbohydrate diet. In some embodiments, the breathing depletes deuterium during sleep. In some embodiments, the breathing depletes deuterium by lymphatic drainage. In some embodiments, the environment filled with vapors having a deuterium level of no more than 135 ppm is prepared by dehumidifying the environment, humidifying with a deuterium-depleted water, or a combination thereof. In some embodiments, the sleeping method comprises use of blue-light blocking glasses or sound therapy or a combination thereof. In some embodiments, the sleeping method increases a melatonin level in the subject. In some embodiments, the cold and hot thermotherapy or thermogenesis burns adipose tissue of the subject. In some embodiments, the cold and hot thermotherapy or thermogenesis enhances sleep length or REM cycle length in the subject. In some embodiments, the at least one of acupuncture, chiropractic manipulation, and massage therapy improved function of lymphatics or a digestive system of the subject. In some embodiments, the ozone and hyperbaric oxygen therapy provides an oxygen level of greater than 21%. In some embodiments, applying the topical composition on the skin decreases a deuterium level of the skin. In some embodiments, applying the topical composition on the skin decreases a deuterium level of skin microbiome. In some embodiments, administering washes and cleanses decreases a deuterium level of a skin of the subject. In some embodiments, administering washes and cleanses decreases a deuterium level of skin microbiome. In some embodiments, the deuterium-depleting protocol is used in combination with an anti-cancer agent. In some embodiments, the deuterium-depleting protocol is used in combination with a conventional therapy for the metabolic disease. In some embodiments, the method prevents weight loss. In some embodiments, the method prevents in weight gain.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 shows comparison of metabolic profile changes associated with natural deuterium depletion by low deuterium fatty acid oxidation and low deuterium metabolic water recycling from the mitochondrial matrix during citrate, isocitrate and malate formation; the target of fumarate hydratase activation and hyperbaric oxygen treatment combined with a ketogenic diet.

FIG. 2 shows transverse and sagittal plane MRI scan images of the brain of a human subject.

FIG. 3 shows resting metabolic rate in human subjects after a week of consuming deuterium depleted water.

FIG. 4A shows the tissue deuterium level measured at day 1, 27, and 63.

FIG. 4B shows ATP production measured at day 1, 27, and 63.

FIG. 4C shows inflammatory fluid decrease at day 0, 7, and 14.

FIG. 4D shows the basal metabolic rate at days 1, 7, 14, 27, 42, and 63.

FIG. 4E shows the breath hold—morning control pause at day 0, 7, 14, 27, and 42.

FIG. 4F shows the grip strength of right and left hands at days 0, 7, 14, 27, 42, and 63.

FIG. 4G shows the fine motor speed of right and left hands at days 0, 7, 14, 27, and 63.

### DETAILED DESCRIPTION

Deuterium, also referred herein as deuteron, is a less common, stable isotope of hydrogen. Protium is the most common hydrogen isotope and also commonly referred to as hydrogen. Deuterium introduces a mass increase of 100% as compared to protium by being composed of two undivided atomic nuclear constituents, a single proton and a single neutron. The doubling of mass in deuterium as compared to a hydrogen provides a way to differentially detect the two atoms. In corporation of deuterium in place of a hydrogen in various biological molecules may induce uncontrolled cellular proliferation and cause structural damage during molecule synthesis and energy transfer among other effects. The biological molecules with incorporated with deuterium may result in heavier and more viscous environment, which may reduce the mitochondrial function and may slow the nanomotors in the mitochondria that generate energy in the form of ATP. As such, reducing deuterium levels may provide an approach to enhance efficiency of ATP production, cellular energy and metabolism.

Deutenomics deal with fundamental and basic autonomic events in biology that separate and/or discriminate deuterons from protons before any autonomic biological reaction architecture may normally ensue. The structure and function in organic molecules by bonding, rapid movements, specific resonances and collective tunneling involve a proton, or a hydrogen nucleus. Application of deutenomics in medicine for deuterium discrimination may have in broad applications to prevent, diagnose, treat and manage diseases, as well as to maintain strength and health. Deutenomics may guide autonomic biological energy transfer and molecule synthesis reactions by discrimination of deuterium in order to avoid cellular damage. When deutenomic processes fail, deuterium may become enriched in cells, where they can impose a proliferating phenotype with less differentiated cellular functions. As such, protocols to decrease the deuterium levels in a subject below 130 parts per million (ppm) may provide a tool to treat various metabolic diseases and disorders and improve health conditions in the subject. Thus, methods to non-invasively measure the deuterium levels in a subject and analyze the measurements can provide valuable and actionable information about the health and condition of the subject.

Provided herein are methods to measure and lower deuterium levels in a subject. The subject may be administered one or more of various deuterium depletion protocols described herein designed to lower the deuterium level in the subject. Before, during, and after the deuterium depletion protocol, various biological samples are collected from the subject in a non-invasive or minimally invasive manner. The biological sample may include one or more of exhaled breath condensate, urine, sweat, tears, blood, plasma, serum, stool, spinal fluid, DNA, RNA, isolated nucleic acids, or cell extract. The deuterium levels of the samples are measured to determine the deuterium levels at different time points. This can help track the progress of deuterium depletion, or the deuterium depletion rate, in the subject and their health conditions. Deuterium levels can be detected and measured by various laboratory deuterium detection methods, including but not limited to laser spectroscopy, mass spectrometry, and other laboratory chemistry methods. Alternatively or in combination, the subject may undergo altered proton magnetic resonance (MRI) before, during, and after the deuterium depletion protocol to detect the deuterium levels in a tissue of interest. Use of deuterium depletion and/or discrimination protocols may provide improved cellular functionality in subjects, improved cellular functions, and reduces cellular dysfunctions and various related cascade health effects. The methods described herein provides various advantages, including the capability to non-invasively and repeatedly measuring deuterium levels in the subject and the ability to generate a more accurate indicator of the health and condition of the subject.

Provided herein are methods of changing the abundance of natural isotopes by providing a deuterium depletion protocol to a subject and observing the variations of deuterium in metabolites taken from biological samples, such as bodily fluids. Mitochondrial deuterium and stable isotope depletion via water processing during biochemical metabolic drying, such as glycolysis, and metabolic hydration reactions of the TCA cycle in biological systems alters natural isotope distribution and provides biomarkers of metabolic health. Deuterium levels can be measured for the assessment of metabolic health and diagnosis of metabolic diseases. Changing deuterium burden (enrichment) in body fluids in various nutritional stages provides biomarkers for drug dosing, individual variations in metabolism, stable isotope enrichment of tissues and body fluids, toxicology, ATP dependent proton transfer processes, energy production and carbon balance for weight loss and health maintenance.

Disclosed herein are methods of measuring a deuterium level to determine a rate of deuterium depletion in a subject comprising: a) obtaining a first biological fluid from the subject; b) measuring an initial level of deuterium in the first biological fluid; c) providing a deuterium-depletion protocol to the subject; d) obtaining a subsequent biological fluid from the subject; and e) measuring a subsequent level of deuterium in the subsequent biological fluid, wherein the initial level and the subsequent level are used to determine a rate of deuterium depletion in the subject.

Described herein are methods of measuring a deuterium level in a subject comprising: a) measuring an initial deuterium level in a tissue of interest the subject; b) providing a deuterium-depletion protocol to the subject; c) measuring a subsequent deuterium level in the tissue of interest in the subject, wherein the initial deuterium level and the subsequent deuterium level are used to determine a rate of deuterium depletion level in the subject, wherein the measuring of steps a) and c) comprises measuring deuterium level in the tissue of interest using MRI.

Provided herein are methods of treating a metabolic disease in a subject comprising: a) measuring an initial deuterium level in a first biological fluid from the subject; b) providing a deuterium-depletion protocol to the subject; c) measuring a subsequent deuterium level in a subsequent biological fluid, wherein the initial deuterium level and the subsequent deuterium level are used to determine a status of the metabolic disease in the subject.

Deuterium may be stored in various organelles, including mitochondria, or biomolecules, such as polypeptides, that are operationally or physically connected to nanomotors responsible for generating ATP. The mitochondria, organelles, or biomolecules may act as governors to slow down the rotational speed of the nanomotors, including but not limited to ATP synthase. Such governors work by storing or binding deuterium and slowing down the rate of ATP synthase as they get heavier or as their own operation slows down. This slows down the ability to produce ATP and metabolic water but may protect the ATP synthase from being destroyed by decreasing the probability that the ATP synthase are fed a deuterium.

Deutenomics may involve discriminatory biological procedures for structurally and energetically balanced biological systems where the kinetic isotopic properties of deuterium are excessive upon substituting for a proton. As molecule synthesis and energy transfer processes do not select hydrogen out for particular steps in biochemical reaction architectures, rules of binomial distribution determine the isotopic composition of biomolecules based on the random availability of protons and deuterons in nature. In corporation of deuterium in various biological molecules may induce uncontrolled cellular proliferation and cause structural damage during molecule synthesis and energy transfer, in contrast to protons that may be involved in cell differentiation, specialization, cooperation, tissue formation via physiologically regulated molecule synthesis.

Deuterium levels can be measured by its proton relaxation properties using various non-invasive measurement techniques. Proton relaxation properties are affected by deuteronation of water and fat in various body fluids and organs. This is because deuterons compromise collective proton tunneling and movements in response to radio frequency excitation sequences in a magnetic field. Compromised collective proton tunneling due to deuterium accumulation produces low magnetic resonance proton signal density and therefore a decrease in luminescence of individual pixels that form the visual media, i.e. the proton (¹H) nuclear magnetic resonance image, of organs and body fluids. These images are evaluated by radiologists and scientists during diagnostic, screening and health advisory work. Relatively low (1.5 Tesla or lower) energy magnetic diagnostic fields allow deuterium's disruptive effect on proton tunneling indirectly being noticed by the altered proton relaxation properties of biological samples in various diseases using magnetic resonance imaging (MM). The MR images generated by 1.5 T or lower diagnostic magnets in the spin-lattice relaxation property testing mode (basic pulse T1 sequence), may be used to determine tissues specific deuterium saturation states in patients. Proton MM is an indirect tool to measure tissue and body fluid bound deuterium-proton ratios. The MM images can undergo the binary processing to reveal numeric luminescence and color composition values by proton tunneling, i.e. free proton movements, as the function of kinetically strong deuterium isotope effects during the formation of MRI images in disease and health. The deuterium-proton ratio can be used to make decisions regarding various medical or health related protocols and/or administer changes or adjustments in the medical or health related protocols. The deuteronation may be tracked using natural substrates or substrates labeled with an isotope of hydrogen (such as deuterated glucose or deuterated ketones).

Deuterium depleted water (DDW) and natural fatty acids or fats may have less heavy hydrogen isotopes, such as deuterium and/or tritium, than normal water. The relatively high hydrogen content of deuterium depleted water and that of a deuterium-depleted diet, including but not limited to ketogenic, vegan, vegetarian, low-carbohydrate, or paleolithic diet with proper amounts of naturally sourced fat, where hydrogen is the most common atom, may provide good vehicles to deliver deuterium-depleted hydrogen. Such delivery may improve cellular energy transfers, protect cells' structural and functional proteins from deuteration and damage by preserving the integrity of DNA and ATP synthase nanomotors among others. The protium-enriched water and diet may provide an environment more favorable for ATP-producing proteins and structural molecules including cellular DNA and RNA. Decreasing deuterium concentration of the body below 130 ppm has been shown to contribute to delays in the progression of several types of cancer in mice and prolongs their survival. Depletion of body deuterium may be achieved by consumption or prolonged administration of DDW and deuterium-depleted food.

### Measuring Deuterium Levels

Provided herein are methods of measuring deuterium levels in a sample from a subject. The deuterium level in the sample from the subject is measured using non-invasive methods described herein to allow for multiple or repeated measurements from the subject. In some embodiments, the deuterium level is measured using laser spectroscopy. In some embodiments, the deuterium level is measured using water-based laser spectroscopy. In some embodiments, the deuterium level is measured using mass spectrometry. In some embodiments, the deuterium level is measured using isotope ratio mass spectrometry. In some embodiments, the deuterium level is measured using other analytical devices such as a mass spectrophotometer, gas spectrophotometer, or any other measuring tool that can quantitate hydrogen isotopes. In some embodiments, the deuterium level is measured using MRI. In some embodiments, the deuterium level is measured by MRI tissue luminescence. In some embodiments, the deuterium level is measured by proton (1H) nuclear MRI of the tissue of interest in the subject.

In some embodiments, the deuterium level is measured using laser spectroscopy. In some embodiments, the deuterium level is measured using water-based laser spectroscopy. In some embodiments, the water-based laser spectroscopy takes simultaneous direct measurement of 2 H/1 H and 18O/16O. In some embodiments, the water-based laser spectroscopy takes direct measurement of 17O/16O stable isotopes. In some embodiments, the water-based laser spectroscopy uses a laser radiation that is coupled to an optical cavity in an off-axis manner and is continuously measured. In some embodiments, the optical cavity provides an extraordinarily long effective optical path length (typically 2-10 km). In some embodiments, the off axis configuration provides robustness and allows for the accurate quantification of water isotopomers with a very high precision.

In some embodiments, the deuterium level is measured using mass spectrometry. In some embodiments, the deuterium level is measured using isotope ratio mass spectrometry.

In some embodiments, the deuterium level is measured using MRI. Generally, the isotopic properties of deuterium-containing water (D₂¹⁶O) are different than light, or only protium-containing water (H₂¹⁶O) and water having a tritium isotope (H₂¹⁸O). These properties are shown in Table 1, which is adapted from Mosin, O. V, Ignatov, I. (2011) Separation of Heavy Isotopes Deuterium (D) and Tritium (T) and Oxygen (¹⁸O) in Water Treatment, Clean Water: Problems and Decisions, Moscow, No. 3-4, pp. 69-78. In some embodiments, MRI uses the mass difference between deuterium and protium, with deuterium having twice the mass of protium and the magnetic field releasing the deuterium and protium at different rates, to distinguish deuterium from protium. In some embodiments, the MRI protocol comprises a T1-weighted sequence using a 1.5-T MM without any contrast agent. In some embodiments, MM images are taken by 1.5-T MRI by a T1 weighted sequence with a contrast agent. In some embodiments, the MRI image provides whiter areas that are indicative of a higher deuterium level than the darker areas. In some embodiments, regions with higher concentrations of deuterium shows up as the whiter areas and corresponded to cancerous tumors. In some embodiments, a MRI image with darker areas correspond to lower deuterium level and a decrease in a cancerous tumor. In some embodiments, the deuterium level is measured by MM tissue luminescence. In some embodiments, the MRI tissue luminescence measurement comprises a luminescence measurement using a standard curve of DDW-filled sample containers. In some embodiments, the standard curve of water with known DDW concentrations is prepared. In some embodiments, the standard curve of water with known DDW concentrations comprises a range of deuterium levels from 25 ppm to 155 ppm. In some embodiments, the standard curve of water with known DDW concentrations comprises deuterium levels of at least two of 155 ppm, 140 ppm, 125 ppm, 110 ppm, 90 ppm, 75 ppm, and 25 ppm. In some embodiments, the water samples for the standard curve are measured at the same time as the subject. In some embodiments, the luminescence from the MRI is plotted to generate a standard curve for analyzing the scan of the subject.

TABLE 1
Properties of water having different hydrogen isotopes

| Physical properties | H₂¹⁶O | D₂¹⁶O | H₂¹⁸ O |
|---|---|---|---|
| Density at 20° C., g/cm³ | 0.997 | 1.105 | 1.111 |
| Temperature of maximum density, ° C. | 3.98 | 11.24 | 4.30 |
| Melting point under 1 atm, ° C. | 0 | 3.81 | 0.28 |
| Boiling point temperature at 1 atm, ° C. | 100.00 | 101.42 | 100.14 |
| The vapor pressure at 100° C., mmHg | 760.00 | 721.60 | 758.10 |
| Viscosity at 20° C., cP | 1.002 | 1.47 | 1.056 |

Provided herein is an exemplary embodiment for analyzing the deuterium level measurements. The deuterium level measured from one or more of urine, saliva, blood, plasma, tear, or sweat sample provides the bodily fluid (BF) deuterium level. In some embodiments, the deuterium level is derived from a ratio of deuterium and protium. In some embodiments, the deuterium level is derived from deuterium concentration. In some embodiments, the deuterium level is provided as ppm of deuterium in the sample. The deuterium level measured from breath condensate (BC) equals 50% BF deuterium level and 50% lungs and heart deuterium. Thus, BC deuterium level provides a marker for the deuterium level in tissues. In some embodiments, deuterium level in at least one of urine, saliva, blood, plasma, tear, or sweat sample=bodily fluid (BF) deuterium level. In some embodiments, deuterium level in a combination of more than one of urine, saliva, blood, plasma, tear, or sweat sample=bodily fluid (BF deuterium level). In some embodiments, the BF deuterium level is taken from the venous blood. In some embodiments, deuterium level in breath condensate (BC) is composed of varying composition of BF deuterium level and deuterium level in the lungs and heart. In some embodiments, deuterium level in BC is taken from 50% BF deuterium level and 50% lungs and heart deuterium level. In some embodiments, the BC deuterium level is a marker for deuterium level in tissue. In some embodiment, the deuterium levels in two different biological fluids from a single subject taken at the same time are within 3 ppm from each other.

In some embodiments, deuterium in breath condensate (BC) is composed of varying composition of BF deuterium level and deuterium level in the lungs and heart. In some embodiments, deuterium level in BC is taken from 0% BF deuterium level and 100% lungs and heart deuterium level. In some embodiments, deuterium in BC is taken from 10% BF deuterium and 90% lungs and heart deuterium. In some embodiments, deuterium in BC is taken from 20% BF deuterium and 80% lungs and heart deuterium. In some embodiments, deuterium in BC is taken from 30% BF deuterium and 70% lungs and heart deuterium. In some embodiments, deuterium in BC is taken from 40% BF deuterium and 60% lungs and heart deuterium. In some embodiments, deuterium in BC is taken from 50% BF deuterium and 50% lungs and heart deuterium. In some embodiments, deuterium in BC is taken from 60% BF deuterium and 40% lungs and heart deuterium. In some embodiments, deuterium in BC is taken from 70% BF deuterium and 30% lungs and heart deuterium. In some embodiments, deuterium in BC is taken from 80% BF deuterium and 20% lungs and heart deuterium. In some embodiments, deuterium in BC is taken from 90% BF deuterium and 10% lungs and heart deuterium. In some embodiments, deuterium in BC is taken from 100% BF deuterium and 0% lungs and heart deuterium. In some embodiments, deuterium in BC is taken from 10% BF deuterium. In some embodiments, deuterium in BC is taken from 10%, 20%, 30%, 40%, 50%, 60%, 70%, 80%, 90%, or 100% BF deuterium. In some embodiments, deuterium in BC is taken from at least 10%, 20%, 30%, 40%, 50%, 60%, 70%, 80%, 90%, or 100% BF deuterium. In some embodiments, deuterium in BC is taken from at most 10%, 20%, 30%, 40%, 50%, 60%, 70%, 80%, 90%, or 100% lungs and heart deuterium. In some embodiments, deuterium in BC is taken from 10%, 20%, 30%, 40%, 50%, 60%, 70%, 80%, 90%, or 100% lungs and heart deuterium. In some embodiments, deuterium in BC is taken from at least 10%, 20%, 30%, 40%, 50%, 60%, 70%, 80%, 90%, or 100% lungs and heart deuterium. In some embodiments, deuterium in BC is taken from at most 10%, 20%, 30%, 40%, 50%, 60%, 70%, 80%, 90%, or 100% lungs and heart deuterium.

In some embodiments, the deuterium level is derived from a ratio of deuterium and protium. In some embodiments, the ratio of deuterium and protium is at least 1:1000, 1:2000, 1:3000, 1:4000, 1:5000, 1:6000, 1:7000, 1:8000, 1:9000, or 1:10000. In some embodiments, the ratio of deuterium and protium is 1:1000, 1:2000, 1:3000, 1:4000, 1:5000, 1:6000, 1:7000, 1:8000, 1:9000, or 1:10000. In some embodiments, the ratio of deuterium and protium is no more than 1:5000, 1:6000, 1:7000, 1:8000, 1:9000, 1:10000, or 1:20000.

In some embodiments, the subject is a human. In some embodiments, the subject is an animal. In some embodiments, the subject is a mammal. In some embodiments, the mammal is a dog. In some embodiments, the mammal is a cat. In some embodiments, the mammal is a horse. In some embodiments, the animal is a bird. In some embodiments, the subject is a plant. In some embodiments, the subject is a food item. In some embodiments, the subject is an animal-based food item. In some embodiments, the subject is a meat product. In some embodiments, the subject is a vegetable. In some embodiments, the subject is a drinkable fluid. In some embodiments, the subject is a drinkable liquid. In some embodiments, the subject is water. In some embodiments, the subject is an organ. In some embodiments, the subject is an organelle. In some embodiments, the subject is a fungus. In some embodiments, the subject is a bacterium. In some embodiments, the subject is a virus. In some embodiments, the subject is a compound.

In some embodiments, the sample is biological fluid. In some embodiments, the biological fluid comprises whole blood, blood, serum, plasma, urine, saliva, breath condensate, tear, or sweat. In some embodiments, the biological fluid comprises whole blood, serum, plasma, urine, saliva, breath condensate, pleural effusions, or mucous discharges. In some embodiments, the biological fluid is breath condensate and blood. In some embodiments, the biological fluid is breath condensate. In some embodiments, the biological fluid is obtained from exhaled breath of the subject. In some embodiments, the biological fluid is treated with a salt to precipitate out the proteins before measuring deuterium levels. In some embodiments, the biological fluid is centrifuged, and the supernatant is collected and measured for deuterium levels.

In some embodiments, the sample is a plant. In some embodiments, the sample is a food item. In some embodiments, the sample is an animal-based food item. In some embodiments, the sample is a meat product. In some embodiments, the sample is a vegetable. In some embodiments, the sample is a drinkable fluid. In some embodiments, the sample is a drinkable liquid. In some embodiments, the sample is water. In some embodiments, the sample is a supplement. In some embodiments, the sample is a vitamin. In some embodiments, the sample is a mineral. In some embodiments, the sample is treated with a salt to precipitate out the proteins before measuring deuterium levels. In some embodiments, the sample is centrifuged, and the supernatant is collected and measured for deuterium levels. In some embodiments, the sample is an organ. In some embodiments, the sample is an organelle. In some embodiments, the sample is a fungus. In some embodiments, the sample is a bacterium. In some embodiments, the sample is a virus. In some embodiments, the sample is a compound.

In some embodiments, the sample for deuterium level measurement is a tissue of interest. In some embodiments, the tissue of interest comprises at least one of skin, muscle, and adipose tissue. Provided herein are methods of measuring deuterium levels in a subject using MM.

In some embodiments, the sample for MRI measurement is a tissue of interest. In some embodiments, the tissue of interest comprises at least one of skin, muscle, and adipose tissue.

### Deuterium Depletion Protocols

Provided herein are methods of measuring deuterium levels in subjects before and after a deuterium-depletion protocol. In some embodiments, the deuterium-depletion protocol comprises a deuterium depleted nutritional protocol. In some embodiments, the deuterium-depletion protocol comprises plant or animal grown in low deuterium environment. In some embodiments, the deuterium depleted nutritional protocol comprises a diet having a predetermined deuterium content. In some embodiments, the deuterium depleted nutritional protocol comprises providing a ketogenic nutritional protocol to the subject. In some embodiments, the deuterium depleted nutritional protocol comprises a natural plant derived protocol, an animal fat derived protocol, or a combination thereof. In some embodiments, the deuterium depleted nutritional protocol comprises consuming an edible item having about 65 ppm to about 135 ppm deuterium. In some embodiments, the deuterium depleted nutritional protocol comprises consuming an edible item having about 65 ppm to about 125 ppm deuterium. In some embodiments, the deuterium-depletion protocol comprises providing a deuterium-depleted water to the subject. In some embodiments, the deuterium depleted water comprises about 10 ppm to about 135 ppm deuterium. In some embodiments, the deuterium depleted water comprises about 65 ppm to about 125 ppm deuterium. In some embodiments, the deuterium-depletion protocol comprises an anti-cancer agent. In some embodiments, the deuterium-depletion protocol comprises exposure to red or near infrared light. In some embodiments, the deuterium-depletion protocol comprises breathing methods to enhance deuterium depletion. In some embodiments, the deuterium-depletion protocol comprises increasing breath fractionation. In some embodiments, the deuterium-depletion protocol comprises inhaling air having humidity or vapor having a deuterium level below 135 ppm. In some embodiments, the deuterium-depletion protocol comprises inhaling air having humidity or vapor having a deuterium level below 125 ppm. In some embodiments, the deuterium-depletion protocol comprises inhaling air having humidity or vapor having a deuterium level below 115 ppm. In some embodiments, the deuterium-depletion protocol comprises inhaling air having humidity or vapor having a deuterium level below 100 ppm. In some embodiments, the deuterium-depletion protocol comprises sleeping methods to enhance deuterium depletion. In some embodiments, the deuterium-depletion protocol comprises cryotherapy. In some embodiments, the deuterium-depletion protocol comprises ozone and hyperbaric oxygen therapy. In some embodiments, the deuterium-depletion protocol comprises injection of a composition having a deuterium level below 135 ppm. In some embodiments, the deuterium-depletion protocol comprises injection of a composition having a deuterium level below 125 ppm. In some embodiments, the deuterium-depletion protocol comprises injection of a composition having a deuterium level below 115 ppm. In some embodiments, the deuterium-depletion protocol comprises injection of a composition having a deuterium level below 100 ppm. In some embodiments, the deuterium-depletion protocol comprises applying a composition having a deuterium level below 135 ppm. In some embodiments, the deuterium-depletion protocol comprises applying a composition having a deuterium level below 125 ppm. In some embodiments, the deuterium-depletion protocol comprises applying a composition having a deuterium level below 115 ppm. In some embodiments, the deuterium-depletion protocol comprises applying a composition having a deuterium level below 100 ppm. In some embodiments, the deuterium-depletion protocol comprises undergoing washes and cleanses with a fluid having a deuterium level below 135 ppm. In some embodiments, the deuterium-depletion protocol comprises undergoing washes and cleanses with a fluid having a deuterium level below 125 ppm. In some embodiments, the deuterium-depletion protocol comprises undergoing washes and cleanses with a fluid having a deuterium level below 115 ppm. In some embodiments, the deuterium-depletion protocol comprises undergoing washes and cleanses with a fluid having a deuterium level below 100 ppm. In some embodiments, the washes of the deuterium-depletion protocol comprise washing the ear, eye, nose, colon, vagina, or penis of the subject. In some embodiments, the deuterium-depletion protocol comprises cold and hot thermotherapy or thermogenesis. In some embodiments, the deuterium-depletion protocol comprises sound therapy. In some embodiments, the deuterium-depletion protocol comprises at least one of acupuncture, chiropractic manipulation, and massage therapy.

In certain embodiments, the deuterium-depletion protocol is a diet with deuterium level of no more than 135 ppm. In some embodiments, the diet is at least one of a vegan diet, a vegetarian diet, a ketogenic diet, a low carbohydrate, and a diet with food having a deuterium level of no more than 135 ppm. In some embodiments, the diet comprises an effective level of low deuterium fats. Natural fats may be lower in deuterium than other natural or processed foods. In some embodiments, fats generally have a high number of hydrogens and its isotopes compared to other molecules, where the deuterium may be substituted with hydrogens. In some embodiments, the diet comprises at least one of a dietary supplement, a vitamin supplement, and a mineral supplement. In some embodiments, metabolism of deuterium-depleted fats can lead to at least one of increased ATP production, increased metabolic water production, and creation of biologically active molecules, including but not limited to cholesterol, estrogen, and DNA, that have more favorable three-dimensional structures for biological activity.

In certain embodiments, the deuterium-depletion protocol is exposure to at least one of red and near-infra-red light. In some embodiments, the subject is exposed to the red or near-infra-red light for a pre-determined length of time. In some embodiments, the pre-determined time for exposure to red or near-infra-red light is 1, 5, 10, 20, 30, 40, 50, or 60 minutes. In some embodiments, the pre-determined time for exposure to red or near-infra-red light is 1, 2, 3, 4, 5, 6, 7, 8, 9, 10, 11, or 12 hours. In some embodiments, the pre-determined time for exposure to red or near-infra-red light is at least 1, 5, 10, 20, 30, 40, 50, or 60 minutes. In some embodiments, the pre-determined time for exposure to red or near-infra-red light is at least 1, 2, 3, 4, 5, 6, 7, 8, 9, 10, 11, or 12 hours. In some embodiments, the pre-determined time for exposure to red or near-infra-red light is no more than 1, 5, 10, 20, 30, 40, 50, or 60 minutes. In some embodiments, the pre-determined time for exposure to red or near-infra-red light is no more than 1, 2, 3, 4, 5, 6, 7, 8, 9, 10, 11, or 12 hours. In some embodiments, the subject is exposed to the red or near-infra-red light once. In some embodiments, the subject is exposed to the red or near-infra-red light more than once. In some embodiments, the subject is exposed to the red or near-infra-red light 1, 2, 3, 4, 5, 6, 7, 8, 9, or 10 times for a pre-determined length of time. In some embodiments, the subject is exposed to the red or near-infra-red light at least once a day, once a week, or once a month. In some embodiments, the subject is exposed to the red or near-infra-red light for at least 1, 2, 3, 4, 5, 6, or 7 days. In some embodiments, the subject is exposed to the red or near-infra-red light for at least 1, 2, 3, or 4 weeks. In some embodiments, the subject is exposed to the red or near-infra-red light for at least 1, 2, 3, 4, 5, 6, 7, 8, 9, 10, 11, or 12 months. In some embodiments, the energy from red and near-infra-red light are transferred to the hydrogen-oxygen bonds in water and transported to the intracellular water inside the mitochondria. In some embodiments, this is known as structural water which has a consistency of a metal more than that of a liquid including the fact that it is controlled by electromotive forces and not gravity. The energy transferred from the light may make this structural water much less viscous and, as a consequence, may allow the nanomotors in the mitochondria to turn faster while protecting itself from deuterium resulting in increased ATP and metabolic water production and less destruction of the nanomotors in an environment with an equivalent concentration of deuterium.

In certain embodiments, the deuterium-depletion protocol is breathing methods to enhance deuterium depletion. In some embodiments, the inhaled oxygen binds with the hydrogens from the diet of the subject to make water and CO₂. In some embodiments, the oxygen can bind to deuterium with a higher affinity and avidity than for hydrogen, also referred to as protium. In some embodiment, the ability to increase the oxygen content in the tissue and, possibly, plasma via the breathing methods provided herein can lead to the formation of semi-heavy (DOH) and heavy (D₂O) water that the body can deplete using a variety of ways, including sleep, defecation, and urination.

In certain embodiments, the deuterium-depletion protocol is increasing breath fractionation. In some embodiments, certain biological processes can remove semi-heavy and heavy water from the body, also referred to as deuterium depletion systems. In some embodiments, increasing the effectiveness of one or more of those systems leads to the ability of the body to better fractionate deuterium. In some embodiments, fractionating deuterium describes the ability of the body to remove a greater percentage of deuterium per unit time. In some embodiments, these systems include but are not limited to sleep, glymphatic and lymphatic drainage.

In certain embodiments, the deuterium-depletion protocol is inhaling or breathing humidity, vapors, or mists with a deuterium level of no more than 135 ppm. As an average person typically breathes in 0.5 to 1.5 liters of water from the humidity in the air during sleep. The humidity in the air is typically in a range of about 138 ppm to 151 ppm depending on the location. In some cases, the deuterium-depletion protocol comprises having the subject sleep in a room where the humidity has been lowered using a dehumidifier. In some cases, the deuterium-depletion protocol comprises having the subject sleep in a room where the humidity has been increased with a humidifier using deuterium-depleted water having a deuterium level of no more than 135 ppm. In some cases, the deuterium-depletion protocol comprises having the subject room where the humidity has been lowered using a dehumidifier and then increased with a humidifier using deuterium-depleted water having a deuterium level of no more than 135 ppm. In some embodiments, the deuterium-depletion protocol provided herein results in the subject having a lower amount of deuterium in the body of the subject. In some embodiments, the subject has less deuterium to be remove by natural deuterium depleting systems or by the methods to deplete deuterium described herein after following the breathing protocol described herein.

In certain embodiments, the deuterium-depletion protocol is sleeping methods to enhance deuterium depletion. In some embodiments, the sleeping methods to enhance deuterium depletion is a method that increases the subject's ability to sleep, as measured by at least one of length of sleep, length of time in deep sleep, length of time in REM cycle, and decreases the length of time awake. In some cases, the sleeping methods provided herein increases the subject's ability to deplete deuterium. In some embodiments, the sleeping methods provided herein includes but is not limited to the use of at least one of blue light blocking glasses and sound therapy. In some embodiments, the sleeping methods provided herein comprises using blue light blocking glasses and a sound therapy. In some embodiments, the sound therapy increases the production of melatonin in the subject. In some cases, the increase in levels of melatonin, also known as a sleep hormone, increases the propensity of the subject to fall asleep or sleep for longer length of time or sleep in REM cycle for longer.

In certain embodiments, the deuterium-depletion protocol is cold and hot thermotherapy or thermogenesis. In some embodiments, the cold and hot thermogenesis transfers energy to the hydrogen-oxygen bonds in water, which is transported to the intracellular water inside the mitochondria. In some embodiments, the transported water, also referred to as structural water, has a consistency of a metal more than that of a liquid including the fact that it is controlled by electromotive forces and not gravity. In some cases, the transferred energy makes the structural water less viscous and as a result allows the nanomotors in the mitochondria to turn faster and protect itself from deuterium. In some cases, this can result in increased ATP and metabolic water production and less destruction of the nanomotors in an environment with an equivalent concentration of deuterium. In some cases, the cold and hot thermogenesis can lead to the burning of adipose tissue. In some cases, the cold and hot thermogenesis can lead to a “natural” non-diet related increase in ATP and metabolic water production. In some embodiments, the cold and hot thermotherapy or thermogenesis affect the sleep cycle and length of sleep of the subject, which may lead to more effective deuterium depletion.

In certain embodiments, the deuterium-depletion protocol is sound therapy. In some embodiments, the sound therapy provide energy from the sound waves that are transferred to the hydrogen-oxygen bonds in water and by direct tissue to tissue energy transfer. In some embodiments, the tissue to tissue energy transfer comprises direct fiber to fiber energy transfer or energy transfer among non-aqueous tissue structures. In some embodiments, the energy is transported to the intracellular water inside the mitochondria. In some embodiments, the transported water, also referred to as structural water, has a consistency of a metal more than that of a liquid including the fact that it is controlled by electromotive forces and not gravity. In some embodiments, the energy transferred from the sound waves makes this structural water less viscous and allows the nanomotors in the mitochondria to turn faster while protecting itself from deuterium. In some cases, this may result in increased ATP and metabolic water production and less destruction of the nanomotors in an environment with an equivalent concentration of deuterium.

In certain embodiments, the deuterium-depletion protocol is at least one of acupuncture, chiropractic manipulation, and massage therapy. In some embodiments, the acupuncture, chiropractic manipulation, or massage therapy reduces the obstacles to the energy transfer in the subject. In some embodiments, the acupuncture, chiropractic manipulation, or massage therapy facilitates the energy transfer in the subject. In some cases, the energy transfer is to an intended target in the subject. In some cases, the energy transfer to the intended target in the subject results in additive or synergistic deuterium depletion. In some cases, the additive or synergistic deuterium depletion is achieved by enhancing the natural deuterium depletion systems in the subject that did not have enough energy to work effectively. In some cases, enhancing the natural deuterium depletion systems in the subject is achieved by at least one of enhancing the sleep of the subject, opening of the lymphatics and glymphatics, and facilitating the function of the digestive system and its activities.

In certain embodiments, the deuterium-depletion protocol is ozone and hyperbaric oxygen therapy (HBOT). In some cases, the ozone and HBOT provide mechanical ways to increase the oxygen in tissues and plasma without changing the breathing technique of the subject. In some cases, the ozone and HBOT is performed under a pre-determined pressure that increases the oxygen available to the subject for intake. In some cases, the ozone and HBOT provides increased oxygen availability to tissues of the subject. In some cases, the ozone and HBOT increases oxygen availability than available without the ozone and HBOT. In some cases, the ozone and HBOT increases oxygen available to tissues of a subject having a disease or disorder or disease as compared to without the ozone and HBOT. In some cases, the oxygen that is forced into the plasma and tissue binds with the hydrogen and other molecules from the subject's diet to make water and CO₂ molecules. In some cases, the oxygen binds to deuterium with a higher affinity and avidity than hydrogen, also referred to as protium. In some embodiment, the ability to increase the oxygen content in the tissue and, possibly, plasma via the breathing methods provided herein can lead to the formation of semi-heavy (DOH) and heavy (D₂O) water that the body can deplete using a variety of ways, including sleep, defecation, and urination.

In certain embodiments, the deuterium-depletion protocol is injection of a composition having a deuterium level of no more than 135 ppm. In some cases, the injection of the composition having a deuterium level of no more than 135 ppm includes but is not limited to injection directly into tissue of interest or into a blood vessel to carry the composition to the tissue of interest. In some cases, the composition having a deuterium level of no more than 135 ppm is deuterium-depleted water. In some cases, the tissue of interest has a high deuterium level. In some cases, the injection results in a decrease in the deuterium level in the tissue of interest treated with the deuterium-depleted composition. In some cases, the injection results in an increase in at least one of ATP, metabolic water, and effective regulatory molecules. In some cases, the increase in at least one of ATP, metabolic water, and effective regulatory molecules from the injection provides the treated tissue with increased cellular energy to facilitate healing, enhanced efficiency in cellular processes, or enhanced tissue and organ function. In some cases, the tissue of interest for treatment include but is not limited to gut, brain, heart, or tumor.

In some embodiments, the deuterium-depletion protocol can be administered by any route suitable for the administration, such as, for example, topical, dermal, subcutaneous, intraperitoneal, intravenous, intramuscular, or oral. In some embodiments, the deuterium-depletion protocol is administered orally. In some embodiments, the deuterium-depletion protocol is administered intravenously. In some embodiments, the deuterium-depletion protocol is administered topically.

In certain embodiments, the deuterium-depletion protocol is applying a topical composition having a deuterium level of no more than 135 ppm on skin. In some embodiments, the topical composition is a gel, a cream, or a lotion. In some embodiments, the application of the topical composition results delivery of the composition onto the applied tissue or the area near the applied tissue, or penetrating into and beneath the applied tissue. In some embodiments, the application decreases the deuterium level in or around the applied tissue, especially in tissues having a high deuterium level. In some embodiments, the application results in an increase in ATP, metabolic water, and effective regulatory molecules. In some embodiments, the increase in at least one of ATP, metabolic water, and effective regulatory molecules from the application provides the treated tissue with increased cellular energy to facilitate healing, enhanced efficiency in cellular processes, or enhanced tissue and organ function. In some embodiments, the application results in delivery of the composition to the bacteria on the skin. In some embodiments, the application affects the deuterium levels of the skin microbiome. In some embodiments, the topical composition is applied in combination with an application of an electric current to enhance the penetration of the composition into the skin or skin microbiome. In some embodiments, the application of the topical composition in combination with the application of an electric current provides for penetration of the deuterium-depleted topical composition into deeper layers of the skin, tissue, or organ. In some embodiments, the application of the topical composition in combination with the application of an electric current provides for penetration of the deuterium-depleted topical composition into the bacterial cells comprising the microbiome. In some embodiments, the topical composition is applied in combination with an application of a sound wave to enhance the penetration of the composition into the skin or skin microbiome. In some embodiments, the application of the topical composition in combination with the application of a sound wave provides for penetration of the deuterium-depleted topical composition into deeper layers of the skin, tissue, or organ. In some embodiments, the application of the topical composition in combination with the application of a sound wave provides for penetration of the deuterium-depleted topical composition into the bacterial cells comprising the microbiome. In some embodiments, the topical composition is applied in combination with an application of a light wave to enhance the penetration of the composition into the skin or skin microbiome. In some embodiments, the application of the topical composition in combination with the application of a light wave provides for penetration of the deuterium-depleted topical composition into deeper layers of the skin, tissue, or organ. In some embodiments, the application of the topical composition in combination with the application of a light wave provides for penetration of the deuterium-depleted topical composition into the bacterial cells comprising the microbiome. In some embodiments, the topical composition is applied in combination with an application of a vibration to enhance the penetration of the composition into the skin or skin microbiome. In some embodiments, the application of the topical composition in combination with the application of a vibration provides for penetration of the deuterium-depleted topical composition into deeper layers of the skin, tissue, or organ. In some embodiments, the application of the topical composition in combination with the application of a vibration provides for penetration of the deuterium-depleted topical composition into the bacterial cells comprising the microbiome. In some embodiments, the vibration is a mechanical vibration. In some embodiments, the vibration is a sonic vibration.

In some embodiments, the application affects the deuterium levels of the skin cells, including but not limited to keratinocytes. In some embodiments, the application enhances beneficial function of the skin microbiome, including but not limited to increased protective function, regulating function, growth and proliferation of the skin microbiome. In some embodiments, the effect of the application on skin microbiome provides for an enhanced health of the applied tissue. In some embodiments, the effect of the application on skin microbiome provides for an enhanced overall health of the subject.

In certain embodiments, the deuterium-depletion protocol is washing or cleansing a tissue, including but not limited to ear, eyes, nose, colon, vagina, or penis, with a composition having a deuterium level of no more than 135 ppm. In some embodiments, washing or cleansing with the composition provides for delivery of the composition onto the applied tissue or the area near the applied tissue, or penetrating into and beneath the applied tissue. In some embodiments, the washing or cleansing decreases the deuterium level in or around the applied tissue, especially in tissues having a high deuterium level. In some embodiments, the washing or cleansing results in an increase in ATP, metabolic water, and effective regulatory molecules. In some embodiments, the increase in at least one of ATP, metabolic water, and effective regulatory molecules from the washing or cleansing provides the treated tissue with increased cellular energy to facilitate healing, enhanced efficiency in cellular processes, or enhanced tissue and organ function.

In some embodiments, the washing or cleansing results in delivery of the composition to the bacterial and viral load on the skin. In some embodiments, the washing or cleansing provides for lead to the more effective operation, growth or intended biological purpose and increase the overall health of the individual or, at a minimum, the health of the applied area. In some embodiments, the washing or cleansing enhances beneficial function of bacterial and viral load on the skin, including but not limited to increased protective function, regulating function, regulating growth and proliferation of the skin bacterial and viral load. In some embodiments, the washing or cleansing decreases unwanted bacterial or viral overgrowth.

In some embodiments, the deuterium depleted nutritional protocol comprises eating edible goods having about 65 ppm to about 125 ppm deuterium. In some embodiments, the deuterium depleted nutritional protocol comprises eating edible goods having about 65 ppm to about 120 ppm deuterium. In some embodiments, the deuterium depleted nutritional protocol comprises eating edible goods having about 65 ppm to about 115 ppm deuterium. In some embodiments, the deuterium depleted nutritional protocol comprises eating edible goods having about 65 ppm to about 110 ppm deuterium. In some embodiments, the deuterium depleted nutritional protocol comprises eating edible goods having about 65 ppm to about 100 ppm deuterium. In some embodiments, the deuterium depleted nutritional protocol comprises eating edible goods having about 65 ppm to about 90 ppm deuterium. In some embodiments, the deuterium depleted nutritional protocol comprises eating edible goods having about 65 ppm to about 80 ppm deuterium. In some embodiments, the deuterium depleted nutritional protocol comprises eating edible goods having about 65 ppm deuterium. In some embodiments, the deuterium depleted nutritional protocol comprises eating edible goods having about 0 ppm to about 125 ppm deuterium. In some embodiments, the deuterium depleted nutritional protocol comprises eating edible goods having about 10 ppm to about 125 ppm deuterium. In some embodiments, the deuterium depleted nutritional protocol comprises eating edible goods having about 20 ppm to about 125 ppm deuterium. In some embodiments, the deuterium depleted nutritional protocol comprises eating edible goods having about 30 ppm to about 125 ppm deuterium. In some embodiments, the deuterium depleted nutritional protocol comprises eating edible goods having about 40 ppm to about 125 ppm deuterium. In some embodiments, the deuterium depleted nutritional protocol comprises eating edible goods having about 50 ppm to about 125 ppm deuterium. In some embodiments, the deuterium depleted nutritional protocol comprises eating edible goods having about 60 ppm to about 125 ppm deuterium. In some embodiments, the deuterium depleted nutritional protocol comprises eating edible goods having about 65 ppm to about 125 ppm deuterium. In some embodiments, the deuterium depleted nutritional protocol is administered in combination with another nutritional protocol.

In some embodiments, the deuterium depleted water has about 65 ppm to about 125 ppm deuterium. In some embodiments, the deuterium depleted water has about 65 ppm to about 120 ppm deuterium. In some embodiments, the deuterium depleted water has about 65 ppm to about 115 ppm deuterium. In some embodiments, the deuterium depleted water has about 65 ppm to about 110 ppm deuterium. In some embodiments, the deuterium depleted water has about 65 ppm to about 100 ppm deuterium. In some embodiments, the deuterium depleted water has about 65 ppm to about 90 ppm deuterium. In some embodiments, the deuterium depleted water has about 65 ppm to about 80 ppm deuterium. In some embodiments, the deuterium depleted water has about 65 ppm deuterium. In some embodiments, the deuterium depleted water has about 0 ppm to about 125 ppm deuterium. In some embodiments, the deuterium depleted water has about 10 ppm to about 125 ppm deuterium. In some embodiments, the deuterium depleted water has about 20 ppm to about 125 ppm deuterium. In some embodiments, the deuterium depleted water has about 30 ppm to about 125 ppm deuterium. In some embodiments, the deuterium depleted water has about 40 ppm to about 125 ppm deuterium. In some embodiments, the deuterium depleted water has about 50 ppm to about 125 ppm deuterium. In some embodiments, the deuterium depleted water has about 60 ppm to about 125 ppm deuterium. In some embodiments, the deuterium depleted water has about 65 ppm to about 125 ppm deuterium.

In some embodiments, the deuterium depleted composition has a deuterium level of no more than 135 ppm. In some embodiments, the deuterium depleted composition is a gel, a cream, or a lotion. In some embodiments, the deuterium depleted composition is a food item. In some embodiments, the deuterium depleted composition has about 65 ppm to about 125 ppm deuterium. In some embodiments, the deuterium depleted composition has about 65 ppm to about 120 ppm deuterium. In some embodiments, the deuterium depleted composition has about 65 ppm to about 115 ppm deuterium. In some embodiments, the deuterium depleted composition has about 65 ppm to about 110 ppm deuterium. In some embodiments, the deuterium depleted composition has about 65 ppm to about 100 ppm deuterium. In some embodiments, the deuterium depleted composition has about 65 ppm to about 90 ppm deuterium. In some embodiments, the deuterium depleted composition has about 65 ppm to about 80 ppm deuterium. In some embodiments, the deuterium depleted composition has about 65 ppm deuterium. In some embodiments, the deuterium depleted composition has about 0 ppm to about 125 ppm deuterium. In some embodiments, the deuterium depleted composition has about 10 ppm to about 125 ppm deuterium. In some embodiments, the deuterium depleted composition has about 20 ppm to about 125 ppm deuterium. In some embodiments, the deuterium depleted composition has about 30 ppm to about 125 ppm deuterium. In some embodiments, the deuterium depleted composition has about 40 ppm to about 125 ppm deuterium. In some embodiments, the deuterium depleted composition has about 50 ppm to about 125 ppm deuterium. In some embodiments, the deuterium depleted composition has about 60 ppm to about 125 ppm deuterium. In some embodiments, the deuterium depleted composition has about 65 ppm to about 125 ppm deuterium.

In some embodiments, the deuterium-depletion protocol comprises an anti-cancer agent. In some embodiments, the anti-cancer agent is a chemotherapeutic. In some embodiments, the anti-cancer agent is antineoplastic agent. In some embodiments, the antineoplastic agent is capable of acting to prevent, inhibit, or halt the development of a cancer a tumor or a neoplasm. In some embodiments, the antineoplastic agents include, but are not limited to, antimetabolites, biological response modifiers, bleomycins, DNA alkylating agents, DNA cross-linking agents, enzymes, hormones, monoclonal antibodies, platinum complexes, proteasome inhibitors, taxanes, vincas, topoisomerase inhibitors, tyrosine kinase inhibitors, nucleoside analogues, antifolates, anthracyclines, podophyllotoxins, alkylating agents, mTOR inhibitors, retinoids, histone deacetylase inhibitors, and immunomodulatory agents.

Provided herein are methods of administering a deuterium depletion protocol to treat a subject having a disease or a disorder. In certain embodiments, disclosed herein, are methods and compositions useful for the treatment of a disease or a disorder. In some embodiments, the disease or disorder is cancer or a tumor. In some embodiments, the cancer comprises at least one of breast, heart, lung, small intestine, colon, spleen, kidney, bladder, head, neck, ovarian, prostate, brain, pancreatic, skin, bone, bone marrow, blood, thymus, uterine, testicular, liver tumors, or combinations thereof. In some embodiments, the cancer comprises a solid tumor, glioblastoma, stomach cancer, skin cancer, prostate cancer, pancreatic cancer, breast cancer, testicular cancer, thyroid cancer, head and neck cancer, liver cancer, kidney cancer, esophageal cancer, ovarian cancer, colon cancer, lung cancer, lymphoma, or soft tissue cancer. In some embodiments, the cancer is a leukemia. In some embodiments, the cancer is chronic lymphocytic leukemia.

In certain embodiments, disclosed herein, are methods and compositions useful for the treatment of a disease or a disorder. In some embodiments, the disease or disorder is a metabolic disease or disorder. In some embodiments, the disease or condition is a cellular disease or disorder. In some embodiments, the metabolic disease or disorder comprises abnormal chemical reactions that affect breakdown of amino acids, carbohydrate, or lipids. In some embodiments, the metabolic disease or disorder comprises abnormal chemical reactions that affect the mitochondria. In some embodiments, the metabolic disease or disorder comprises at least one of diabetes, acid-base imbalance, metabolic brain diseases, calcium metabolic disorders, DNA repair-deficiency disorders, glucose metabolism disorders, hyperlactemia, iron metabolism disorders, lipid metabolism disorders, malabsorption syndromes, metabolic syndrome X, inborn error of metabolism, enzyme deficiencies, mitochondrial diseases, phosphorus metabolism disorders, porphyrias, proteostasis deficiencies, metabolic skin diseases, wasting syndrome, and water-electrolyte imbalance. In some embodiments, the metabolic disease or disorder comprises acidosis, alkalosis, anorexia, weight loss, morbid obesity, Prader-Willi syndrome, Type I diabetes, Type II diabetes, sleep apnea, insomnia, adrenal fatigue, lymes disease, autoimmune disorders, cognitive decline, infertility, Parkinson's Disease, drug dependency, alcohol dependency, traumatic brain injury, depression, chronic fatigue, post-traumatic syndrome disease, Asperger's syndrome, ADHD, childhood epilepsy, cardiovascular disease, hormone imbalance, high cholesterol, hyperglycemia, lipoma, pain, and anxiety. In some embodiments, the metabolic disease or disorder is diabetes. In some embodiments, the metabolic disease or disorder is Type I diabetes. In some embodiments, the metabolic disease or disorder is Type II diabetes. In some embodiments, the metabolic disease or disorder is a mitochondrial disease. In some embodiments, the metabolic disease or disorder results in premature aging or poor athletic performance. In some embodiments, administration of the methods described herein may improve premature aging, extend lifespan, or improve athletic performance.

In some embodiments, the deuterium-depletion protocol is administered on a suitable schedule, for example, once a day, twice a day, 3 times a day, 4 times a day, 5 times a day, 6 times a day, 7 times a day, 8 times a day, 9 times a day, or 10 times a day. In some embodiments, the deuterium-depletion protocol is administered at least once a day, twice a day, 3 times a day, 4 times a day, 5 times a day, 6 times a day, 7 times a day, 8 times a day, 9 times a day, or 10 times a day. In some embodiments, the deuterium-depletion protocol is administered no more than once a day, twice a day, 3 times a day, 4 times a day, 5 times a day, 6 times a day, 7 times a day, 8 times a day, 9 times a day, or 10 times a day. In some embodiments, the deuterium-depletion protocol is administered daily, once every two days, once every three days, once every four days, once every five days, once every six days, weekly, twice weekly, monthly, twice monthly, once every two weeks, once every three weeks, or once every four weeks. In some embodiments, the deuterium-depletion protocol is administered at least daily, once every two days, once every three days, once every four days, once every five days, once every six days, weekly, twice weekly, monthly, twice monthly, once every two weeks, once every three weeks, or once every four weeks. The deuterium-depletion protocol can be administered in any therapeutically effective amount.

In some embodiments, the deuterium level is measured on a suitable schedule. In some embodiments, the deuterium level is measured after 1, 2, 3, 4, 5, 6, 7, 8, 9, 10, 11, 12, 13, 14, 15, 16, 17, 18, 19, 20, 21, 22, 23, or 24 hours after first administration of the deuterium depletion protocol. In some embodiments, the deuterium level is measured after 1, 2, 3, 4, 5, 6, or 7 days after first administration of the deuterium depletion protocol. In some embodiments, the deuterium level is measured after 1, 2, 3, 4, 5, 6, 7, 8, 9, 10, 11, or 12 weeks after first administration of the deuterium depletion protocol. In some embodiments, the deuterium level is measured between about 1 hours and about 24 hours after first administration of the deuterium depletion protocol. In some embodiments, the deuterium level is measured between about 1 day and about 7 days after first administration of the deuterium depletion protocol. In some embodiments, the deuterium level is measured between about 1 week and about 8 weeks after first administration of the deuterium depletion protocol. In some embodiments, the deuterium level is measured at least once a day, twice a day, 3 times a day, 4 times a day, 5 times a day, 6 times a day, 7 times a day, 8 times a day, 9 times a day, or 10 times a day after first administration of the deuterium depletion protocol. In some embodiments, the deuterium level is measured at least daily, once every two days, once every three days, once every four days, once every five days, once every six days, weekly, twice weekly, monthly, twice monthly, once every two weeks, once every three weeks, or once every four weeks after first administration of the deuterium depletion protocol. In some embodiments, the deuterium level is measured periodically based on the metabolic disease or disorder, the clinical status of the subject, or concomitant therapies.

In some embodiments, the deuterium level is measured after 1, 2, 3, 4, 5, 6, 7, 8, 9, 10, 11, 12, 13, 14, 15, 16, 17, 18, 19, 20, 21, 22, 23, or 24 hours after last administration of the deuterium depletion protocol. In some embodiments, the deuterium level is measured after 1, 2, 3, 4, 5, 6, or 7 days after last administration of the deuterium depletion protocol. In some embodiments, the deuterium level is measured after 1, 2, 3, 4, 5, 6, 7, 8, 9, 10, 11, or 12 weeks after last administration of the deuterium depletion protocol. In some embodiments, the deuterium level is measured between about 1 hours and about 24 hours after last administration of the deuterium depletion protocol. In some embodiments, the deuterium level is measured between about 1 day and about 7 days after last administration of the deuterium depletion protocol. In some embodiments, the deuterium level is measured between about 1 week and about 8 weeks after last administration of the deuterium depletion protocol. In some embodiments, the deuterium level is measured at least once a day, twice a day, 3 times a day, 4 times a day, 5 times a day, 6 times a day, 7 times a day, 8 times a day, 9 times a day, or 10 times a day after last administration of the deuterium depletion protocol. In some embodiments, the deuterium level is measured at least daily, once every two days, once every three days, once every four days, once every five days, once every six days, weekly, twice weekly, monthly, twice monthly, once every two weeks, once every three weeks, or once every four weeks after last administration of the deuterium depletion protocol.

Described herein are various deuterium depletion protocols that may increase ATP production. In some embodiments, ATP production is measured by revolution of the ATP synthase to determine its effect from a deuterium depletion protocol. In some embodiments, the ATP synthase's F₀F₁ C protein nanomotor's revolutions per minute (RPM) in micromoles/min/mg units is measured. In some cases, body fluid-derived metabolic and environmental deuterium levels are compared with metabolic water ²H content as a low deuterium depleted diet standard. In some cases, body fluid (mostly environmental) and mitochondrial matrix (mostly metabolic) water deuterium contents are different, thus ATP-RPM idling values of 15% to 30% from body fluid assessments are common.

Provided herein are various deuterium depletion protocols that may increasethe metabolic water production. In some cases, the metabolic water production measured by calculation as follows: Each water molecule has two hydrogens. For each day consisting of 86400 seconds, a human body transfers about 5.6×10²⁴×86400=4.83×10²⁹ protons daily. The 4.83×10²⁹/protons yield/2 =2.42×10²⁹ water molecules, where 2.42×10²⁹/6.022×10²³=401727 mol water. For 1 mol water=18 g, 401727×18=7231086 g of water that is recycled in mitochondria each day, which is the equivalent of 7231 liter (1910 gallons) of water per day. In some embodiments, a molecule that stops the recycling of water, including but not limited to deuterium breaking a nanomotor, would stop such metabolic water from being made.

Described herein are various deuterium depletion protocols that may result in improved cellular function. In some embodiments, the deuterium depletion protocols provided herein affect the resting metabolic rate, the basal amount of energy required for a body to function for a day. In some embodiments, the deuterium depletion protocols provided herein increase the subject's resting metabolic rate by more than 10%, 20%, 30%, 40%, or 50% in less than a week. In some embodiments, the deuterium depletion protocols provided herein increase the subject's resting metabolic rate by more than 10%, 20%, 30%, 40%, or 50% in a week. In some embodiments, the deuterium depletion protocols provided herein increase the subject's resting metabolic rate by more than 10%, 20%, 30%, 40%, or 50% in a month.

### Deutenomics

Water that contains deuterium, also referred to as heavy water, may have chemical properties that differ from regular water having hydrogen. This difference in water with deuterium may be used in nuclear technology. The biological significance of deuterium discrimination by filtering it from hydrogen by cells and their protein nanomotor machinery in various organelles has not been explored for medicine and health. Deutenomics describes this process.

The atomic principle that builds organic molecular structures, while transferring chemical energy by movements, vibrations, tunneling and resonance for many life related metabolic and structural events, is the proton, or hydrogen (¹H) nucleus, with an undivided mass of 1 Dalton. A stable isotope of the hydrogen is deuterium, or deuteron, having a mass of 2 Daltons. A deuteron (²H) has both a proton and a neutron. The doubling of mass can provide a series of discriminatory biological procedures in structurally and energetically balanced systems. As molecule synthesis and energy transfer proceeds according to atomic randomization, the molecular synthesis processes and metabolic enzymes may not select hydrogen out from mixtures for particular steps in chemical reaction architectures, binomial distribution principles rule the isotopic composition of biomolecule structures based on the enrichment of protons and deuterons between all reductive and oxidative processes. As deuterons possess isotopic effects, they may drive cellular proliferation and/or structural damage during molecule synthesis, while protons are primarily involved in cell differentiation and biologically controlled synthesis. Deutenomic is a principle that deals with two obligatory autonomic biological processes that first discriminate against a heavy isotope of the same element, for example deuterium needs to be discriminated before proton mediated biological processes and reactions ensue. Deutenomics may have broad applications to govern the prevention, diagnosis, treatment and management of diseases, as well as to maintain strength and health. When deutenomic processes fail, deuterium enrichment increases in cells, by which they switch to a proliferating phenotype with less differentiated cellular functions that can be measured by proton magnetic resonance (MRI) and laboratory chemistry methods.

Adenosine triphosphate (ATP) is a molecule that provides energy to drive many processes in living cells. ATP is formed according to the reaction: ADP+Pi+H+out [symbol] ATP+H₂O+H+in, whereby approximately 70 g ATP decomposes and is produced in each minute in the human body at rest. In the case of strenuous exercise ATP turnover can reach 0.5 kg per minute and in athletes daily ATP production and degradation can reach the weight of their bodies. The synthesis of for example 100,000 g (100 kg; e.g. weightlifting) is equivalent to 197 moles of ATP (ATP=507.18 g/mole), which results in the production of 197×2.5 =492.5 mol metabolic water. This is nearly 8 865 grams, i.e. 8.9 kg (liters) or 2.4 gallons of water per day, which is deuterium free in mitochondria in order to preserve the function and morphology of ATP synthase nanomotors. There are ˜8,000 ATP synthase nanomotors in each side of a single crypt of the mitochondrial matrix. Therefore, both bends of 20 membranes give 320,000 nanomotors per mitochondria. On average, there is about a thousand mitochondria in each cell, yet these vary from 1500-2000 (muscle and liver cells) to 200 (cartilage, skin cells, connective tissues). One human cell weighs about 1 nanogram (1×10⁻⁹ or 0.000000001 g) and an adult man weighs ˜70 kg (70000 g). This indicates that we all have about 70 trillion cells. (7.0×10¹³), yet we use half of this for calculations after correction for cell volume further described elsewhere. In every second, the nanomotors transfer about 500 protons at average speed (3000 rotations per minute), which results in 1.12×1022×500=5.6×1024 protons/second in our body, a process that is dependent on mitochondrial interfacial water viscosity, which is affected by deuterium.

Mitochondrial interfacial water viscosity changes when exposed to electro-magnetic forces, which include light and soundwaves. Although the electromagnetic excitability (absorbance ranges) at specific wavelengths is similar by hydrogen and deuterium the emitted light (such as used by discharge lamps) spectra by hydrogen are notable for their high output in the ultraviolet, with comparatively little output in the visible and infrared, as seen in a hydrogen flame. However, emitted spectra using deuterium have a longer life span and an emissivity (intensity) at the far end of their UV range which is three to five times that of an ordinary hydrogen discharge source, at the same temperature. Deuterium emissions may be a superior light source to the light-hydrogen source for the shortwave UV range. Light emissions can be used as markers of biochemical plasticity that is greatly reduced by deuterium in all rapid hydrogen bonding, moving, tunneling and exchanging biological systems; whereby deuterium related kinetic isotopic differences can be studied and utilized for medicine.

Deuterium depletion plays a key role in maintaining the integrity of the extremely active proton transfer and water producing, as well as intracellular and mitochondrial recycling processes performed by the Hans Krebs/Albert Szent-Györgyi cycle.

There are additional staggering numbers when the “distance” protons collectively travel on the surface of the spinning mitochondrial nanomotors in each second during energy production is accounted for. Each nanomotor has a 5 nanometer spinning F1 protein assembly in diameter; hence their name nanomotor. A human body has close to 1.12×10²² nanomotors that spin 50 times per second with their 15.7 nanometers in perimeter (5 nm diameter×3.14 (Pi)), which results in a 785 nanometer per second in distance. When multiplied with the number of nanomotors, the result is 8.79×10²⁴ nanometers, which equals with 8.79×10¹⁵ meters, or 8.79×10¹² kilometers, close to a light-year, which is 9.461×10¹² kilometers. It is easy to understand why deuterium, by replacing protons or hydrogens in this delicate and dynamic system, needs to be filtered out for cellular health, compatibility and integrity, where deutenomics can address the autonomic and continuous filtering of deuterium from cellular energy producing processes.

The very rapid, dynamic, multiple and excessive exchanges of deuterium with protons may be mechanisms involved in the process of how differentiated eukaryote biochemical enzyme networks protect themselves against the damaging effects of deuterium to preserve mitochondrial health. Mitochondrial health is key to cellular and human health. Deuterium depletion and the regulation of its exchanges with protons may provide critical processes on how food, drinks, breathing methods, lifestyle, exercise, ointments and/or remedies can be used to maintain, restore or preserve healthy cellular functions.

Human body continuously clears to team from the circulation, with the help of urine and secretory gland functions, or by glycolysis with pentose cycling in circulating blood cells through mitochondria and peroxisomes. The chemical reaction in the cells that replaces deuterium using water's hydrogen, protons or hydroxyl groups is potentially involved in the endless process of deuterium depletion and balance. One deuterium depleting system from circulation is the production and re-absorbance of all, approximately 800 liters primary filtrate in the kidneys, into the circulation by cellular membrane mechanisms in the proximal and distal renal tubes.

Deuterium fractionation in nature using physical processes, in opposition to the continuous biological filtering of deuterium through nanomotors and enzyme reactions, by temperatures and epithelial surface adhesions, may affect bacterial and viral propagation. Kinetic deuterium fractionation is an isotopic fractionation process that separates hydrogen from deuterium by their mass during unidirectional processes. Biological processes are generally unidirectional and are good examples of “kinetic” isotope reactions. As an example, photosynthesis preferentially takes up the light isotope of hydrogen ¹H and carbon ¹²C during assimilation of soil H₂O and atmospheric CO₂ molecules. Kinetic isotope fractionation explains why plant material (and thus fossil fuels, which are derived from plants) is typically depleted in ²H (deuterium) and ¹³C (heavy carbons).

A naturally occurring example of non-biological kinetic fractionation occurs during the evaporation of seawater to form clouds under conditions in which some part of the transport is unidirectional, such as evaporation into very dry air. In this instance, isotopically lighter water molecules (i.e., those with ¹H₂O) may evaporate slightly more easily than the isotopically heavier water molecules ²H₂O. During this process the hydrogen isotopes are fractionated: the clouds become enriched with light water, and the seawater becomes enriched in heavy water. The common cold can be associated with increased fractionation of hydrogen from deuterium in colder air, whereby more deuterium remains at cooler temperatures in epidermal serous or mucinous products, whereby it may support bacterial propagation in epithelial areas like that in the mouth, throat and respiratory mucosal surfaces.

Deuterium Changes (3-D) Metabolic Structures (e.g. Enzymes' Functions and DNA Configurations)

Hydrogen and deuterium ratios in cells can regulate growth signaling, where exceptional kinetic isotope effects and altered collective proton tunneling may be evident by deuterium in hydrogen bonding and bridging physical as well as biological networks. Exceptional deuterium substitution effects may offer explanations for the observation that ²H depletion in water, although being a rare isotope species, possesses anti-cancer properties. Specifically, there are 155 2H2O heavy water molecules out of 1 million ¹H₂O (one ²H₂O out of 6420 ¹H₂O molecules) or 155 ²H atoms out of 1 million ¹H in oceanic water. On the other hand, one part mono-deuterated water ²H¹HO exists in approximately 3210 parts fully protonated surface water, which means there is one deuterium in approximately 3210 water molecules. In biomolecules, e.g. in NADPH and DNA, the two stable isotopes of hydrogen, protium (¹H) and deuterium (²H; D) induce very different physicochemical behaviors. Though ¹²C containing molecules undergo ambient temperature bond-breaking reactions a few percent faster than the ^{D}C counterparts, covalent bonds to protium are typically cleaved >7 times faster than bonds to deuterium because of the large differences in reduced masses and quantum mechanical properties that influence primary kinetic isotope effects. Deuterium in human plasma is abundant with concentrations reaching 12-14 mmol/l, in comparison with calcium's 2.24-2.74 mmol/l, magnesium's 0.75-1.2 mmol/l, potassium's 5.0-5.1 mmol/l and glucose's 3.3-6.1 mmol/l circulating concentrations. Higher magnetic and electronic dipole moments of deuterium may play a role in DNA hydrogen bond stability as well as abnormal cell proliferation. The deuterium/hydrogen (²H/¹H) mass ratio, being the largest among stable isotopes of the same element, may cause major differences in the chemical bonding and collective proton tunneling behaviors ranging from cubic ice to the structural integrity and function of growth signaling proteins, anabolic products of reductive synthesis, such as DNA, RNA and nuclear membrane lipids in newly formed cells.

Lipid molecule may break down into water and carbon dioxide because of the continuous turnover of fatty acids, as well as other substrate product pools, in the cells. Heavy hydrogen in fatty acids may contribute to heavy metabolic water production in mitochondria, therefore, fatty acids that are oxidized in mitochondria may be deuterium depleted for the protection of nanomotor functions. Thus, if cells oxidize fatty acids that are loaded with deuterium, and fatty acids may be synthesized from high deuterium fruit sugars, there may be heavy water formed in mitochondria that break down nanomotors. Fatty acids can be considered as Trojan horses for getting deuterium riding on carbons potentially into the mitochondrial matrix and peroxisomes where metabolic water is formed. Natural ketogenic diets can have primary roles in preserving heart health by preserving mitochondrial functions. Replacing high deuterium containing industrial lipids with that of low deuterium containing natural low-deuterium ones (such as that of grass-fed animals, wild-caught fish, etc.) to be oxidized in mitochondria restores normal proton transferring processes via nanomotor functions, thus ATP production in a balanced, biologically relevant protected manner. Endothelial cells which are exposed to circulating high deuterium cholesterol and fatty acids of industrial source may get damaged, whereby deuterium depleted naturally produced fatty acids have protective effects on the vasculature. Fat consumption enters the TCA cycle as acetyl-CoA.

### Deuterium Compromises Electro-Magnetic Resonance.

The effect of low ²H in water has been shown to control cell proliferation in numerous biological systems in vitro and in vivo. In vitro studies, in which the only difference of the growth media was ²H concentration in water, showed that ²H depletion inhibits cell growth in a dose dependent manner. To the contrary, increasing the ²H concentration over the natural abundance in water can stimulate cell growth. The effects observed can be modeled when the growth rate of tumor cells is significantly inhibited in culture prepared with deuterium-depleted water (DDW) and, importantly, that physically restoring ²H levels by adding heavy water restores cell growth rates. In vivo effectiveness of deuterium depletion has been shown. Complete or partial tumor regression has been established in mice xenografts with MDA-MB-231, MCF-7 human breast adenocarcinoma cells, and PC-3 human prostate tumor cells. When laboratory animals are exposed to the chemical carcinogen(s): 7,12-Dimethylbenz(a)anthracene (DMBA) and cytoplasmic myelocytomatosis oncogene (c-Myc), Ha-ras, and p53 are up-regulated. The consumption of deuterium depletion can suppress the expression of these genes. Deuterium depletion significantly inhibited proliferation of A549 human lung carcinoma cells in vitro, while H460 lung tumor xenografts in laboratory mice showed a 30% growth regression. ²H-depletion has been shown to have anti-cancer effects in a clinical trial on prostate cancer, and a follow up study suggests that ²H-depletion may delay disease progression. Based on these observations, deutenomics offers a promising new modality in cancer treatment and prevention by lowering extra-mitochondrial deuterium loading into cellular DNA. ²H-depletion, in combination to conventional treatments, can improve mean survival in cancer, including in advanced lung cancer with complicated by metastases. In breast cancer patients deuterium depletion treatment, in combination with, or as an extension of, conventional therapies, may significantly improve survival in advanced disease and can be effective in the prevention of recurrences in early stage breast cancer.

### Deuterium Accelerates Bacteria and Viruses to Grow, Replicate and Thrive.

The core reducing equivalent in living cells to produce new DNA and fatty acids via reductive carboxylation and biomolecule synthesis is NADPH and its deuterium loading properties depend on carbon-specific positional glucose phosphate deuterium enrichments, as well as deuterium enrichment of the cytoplasmic and mitochondrial water pools. These mechanisms may be important because intramolecular deuterium distributions reveal disequilibrium and a chemical shift axis for example in fructose-6-phosphate and glucose-6-phosphate. Natural glucose source isolated from leaf starch of common bean (Phaseolus vulgaris) or spinach (Spinacia oleracea) may be depleted in deuterium in the C(2) position. Carbon specific deuterium depletion in fatty acids from plants and other sources is evident, which generate deuterium depleted matrix water in mitochondria during complete oxidation in complex-IV. Variations in carbon specific deuterium content of oxidizable substrates in the pentose and TCA cycles point to the biological role of intramolecular deuterium distributions that may be essential to understand the details of product deuterium abundances in compartmentalized deuterium transferring intracellular systems as oncogenic initiators.

The pentose cycle (oxidative branch) uses water of cytoplasmic origin with higher natural surface water-like deuterium content, which is about 155 parts per million. The full reaction architecture of the irreversible pentose cycle (oxidative branch) may be important as the ring-opening hydrolysis in the pentose cycle results in the production of 6-phospho-D-gluconate, which provides another mole of NADPH to the pentose cycle-deriving cytosolic NADPH pool by phosphogluconate dehydrogenase [EC 1.1.1.43] during the completion of the direct C(1) oxidation process. This reaction is followed by pentose cycling, cytoplasmic water hydrogen exchanges by Lobry de Bruyn-Alberda-van Ekenstein aldose-ketose transformations via glucose-phosphate isomerase (GPI), triose phosphate isomerase (TPI) and channeling into various hexose, pentose and triose phosphate pools that readily mix with carbon specific hydration products of the matrix such as malate, produced by fumarase, using deuterium depleted matrix water in mitochondria. Extensive substrate hydration steps in mitochondria using nutrient-derived hydrogens, as well as the ring opening hydrolysis of the pentose cycle using cytoplasmic water can alter NADH-dependent deuterium enrichments that affect reversible cytosolic NAD+-dependent shuttle systems, including malate dehydrogenase.

The deuterium loading hypothesis of cancer emphasizes that deuterium content of cytoplasmic and mitochondrial water pools are different when they contribute to NADPH synthesis and that even small perturbations in the above mentioned cellular deuterium depleting pathways that use either cytoplasmic or metabolic water in the pentose cycle readily induce aneuploidy, undifferentiated blast cell formation and alteration of nuclear DNA size and function. For example, increased hexose isomerization and pentose cycling with substrate switching from ketogenic palmitate to glucose and glutamine within the pentose and TCA cycles, or simply using deuterium depletion in place of natural abundance water in culture can alter cellular phenotype and proliferation.

²H depletion in water can provide a new adjuvant and protective cancer therapy. The effectiveness of deuterium depletion can be related to Warburg's theory, as it is the product of and preserves healthy mitochondrial function. Matrix deuterium depletion production can prevent irreversible defects in OXPHOS, which may be a trigger for cancer. The role of ²H in biology may help to explain cancer epidemics as it may correlates with excessive ²H loading from processed carbohydrate intake in place of natural fat consumption. The resulting oncometabolites may act as ²H loading substrates (glucose, glutamine serine) or block deuterium depleting gluconeogenesis in the mitochondria as a deuterium depleting carbon processing metabolic hub, which yields DNA with a sugar backbone protected from aneuploidy and instability with healthy hydrogen bonding, tunneling and bridging network. A diet of solid and liquid foods and beverages with deuterium level between 0 and 135 ppm may lower deuterium levels and help to restore cellular function and prevent metabolic dysfunctions.

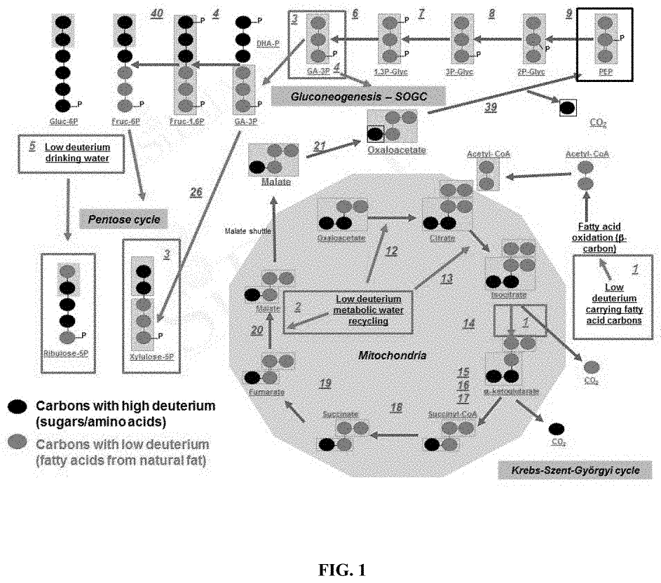

Mitochondrial deuterium and stable isotope depletion via water processing during biochemical metabolic drying, such as glycolysis, and metabolic hydration reactions of the TCA cycle in biological systems alters natural isotope distribution and provides biomarkers of metabolic health as shown in FIG. 1. FIG. 1 shows comparison of metabolic profile changes associated with 1) natural deuterium depletion by low deuterium fatty acid oxidation. Avastin® and Gleevac® exert similar effect and require intact mitochondria for efficacy (boxes No. 1 and 2) low deuterium metabolic water recycling from the mitochondrial matrix during citrate, isocitrate and malate formation; the target of fumarate hydratase activation and hyperbaric oxygen treatment combined with a ketogenic diet (Rd box No. 2). Mitochondrial shuttles, such as the malate shuttle, pass low deuterium carrying fatty acid carbons to gluconeogenesis, where glyceraldehyde-3-phosphate becomes the source of extensive carbon exchange reactions for the non-oxidative pentose cycle to maintain low deuterium saturation in C3′-C5′ pentose sugar carbon positions in RNA and DNA (box No. 3). These are the carbon sites where DNA stability, radiation- and chemotherapy derived hydroxyl radical sensitivities are regulated by hydrogen/deuterium due to primary and secondary intrinsic isotope effects; as well as partially by collective proton tunneling. Besides the C3′-C5′ nucleic acid sugar backbone fragment, de novo nucleic acid base syntheses, hydrogen bonding and deuterium channeling into hydrogen bonds are controlled by the serine oxidation glycine cleavage single carbon cycle pathways. When tumor cells revert to the Warburg phenotype and reductive carboxylation-driven mitochondria, deuterium depletion in free (drinking) water becomes the only deuterium depleting mechanism for specific carbon sites in nucleic acid backbone sugars and the bases.

### Cancer

Cancer may be caused by defective cellular energy production via complete substrate oxidation in mitochondria when protein nanomotors of the matrix become defective due to the damaging effect of deuterium. This is because cellular ATP energy production is a rapid proton (hydrogen) recycling process that is closely accompanied by robust metabolic water production in the mitochondrial matrix, whereby deuterium may replace hydrogen in energy producing proteins. Limiting destructive heavy hydrogen exposures of ATP energy producing moving proteins can improve cellular health and therefore may be considered as an important target to improve medical conditions. Deuterium depletion may protect cellular energy producing and structural proteins from the damaging effects of the heavy isotopes of hydrogen. Depleting hydrogen in water and food using any natural fatty acid as a single compound may achieve this goal in order to restore cellular health, which may improve cancer outcomes.

Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining”, “measuring”, “evaluating”, “assessing,” “assaying,” and “analyzing” are often used interchangeably herein to refer to forms of measurement, and include determining if an element is present or not (for example, detection). These terms can include quantitative, qualitative or quantitative and qualitative determinations. Assessing is alternatively relative or absolute. “Detecting the presence of” includes determining the amount of something present, as well as determining whether it is present or absent.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. The disease can be endometriosis. In some cases, the subject is not necessarily diagnosed or suspected of being at high risk for the disease.

The term “in vivo” is used to describe an event that takes place in a subject's body.

The term “in vitro” is used to describe an event that takes places contained in a container for holding laboratory reagent such that it is separated from the living biological source organism from which the material is obtained. In vitro assays can encompass cell-based assays in which cells alive or dead are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

As used herein the term an “effective amount” refers to the amount of a therapeutic that causes a biological effect when administered to a mammal. Biological effects include, but are not limited to, inhibition or reduction in symptoms, inhibition or reduction in metabolic abnormality, inhibition or reduced tumor growth, reduced tumor metastasis, or prolonged survival of an animal bearing a tumor. A “therapeutic amount” is the concertation of an agent calculated to exert a therapeutic effect. A therapeutic amount encompasses the range of dosages capable of inducing a therapeutic response in a population of individuals. The mammal can be a human individual. The human individual can be afflicted with or suspected or being afflicted with a disease of a condition.

The section headings used herein are for organizational purposes only and are not to be construed as limiting the subject matter described.

### EXAMPLES

The following examples are included for illustrative purposes only and are not intended to limit the scope of the invention.

### Example 1: Breath Condensate Collection for Deuterium Level Measurement

Provided herein is an exemplary embodiment for collecting exhaled breath condensate from a subject for deuterium level measurement. This can be performed using a number of different devices for exhaled breath condensate. In an exemplary embodiment using an exhaled breath condensate collection device Breathy, a metal tube is placed in a freezer for at least three hours to chill the metal tube. The metal tube is then removed from freezer with dry hands and pad and fitted into the exhaled breath condensate collection device. The subject inhales through nose and blows the exhalation through a mouthpiece of the device, being careful not to also inhale through mouth. The subject inhales and exhales into the mouthpiece for 10 minutes using normal breaths. The subject may hear the air flowing out of the exhaled breath condensate collection device. The metal tube is removed while keeping the metal tube upright and placing a blue cap on the open end. Then, the mouthpiece unit is pulled from the metal tube and a cap is placed on the bottom end, making sure the caps are securely fastened on each end of the tube. The capped tube is placed into a sample bag and kept in refrigerator at 4° C. until ready to ship for deuterium measurement.

### Example 2: Breath Condensate Collection for Deuterium Level Measurement

Provided herein is an exemplary embodiment for collecting exhaled breath condensate from a subject for deuterium level measurement. The subject exhales into the breath condensate collector device to collect breath condensates inside the device. The exhaled breath condensate is harvested from the device into a shipping tube. The shipping tube with the exhaled breath condensate is kept in refrigerator at 4° C. until ready to ship for deuterium measurement.

### Example 3: Saliva Collection for Deuterium Level Measurement

Provided herein is an exemplary embodiment for collecting saliva from a subject for deuterium level measurement. 1) The saliva from the subject is collected into a saliva sample tube to about three-fourth full after the foam from the saliva settles. 2) The sample tube is capped and left to stand 20 minutes on a level surface. 3) Then, a pipette bulb outside of the sample tube is squeezed to collect the saliva sample in center of sample tube by releasing bulb. 4) The pipette content is transferred into a second sample tube by squeezing bulb. 5) Steps 3 and 4 are repeated until the second sample tube is filled to a ribbed line. 6) The second sample tube is capped tightly and shaken vigorously for 20 seconds. 7) The second sample tube with saliva is placed in a sample bag and kept in refrigerator at 4° C. until ready to ship for deuterium measurement.

### Example 4: Urine Collection for Deuterium Level Measurement

Provided herein is an exemplary embodiment for collecting urine from a subject for deuterium level measurement. 1) The urine is collected midstream in a clean vessel, such as a paper sample cup or a plastic sample cup. 2) The sample cup is left to stand 20 minutes on a level surface. 3) Then, a pipette bulb outside of the sample cup is squeezed to collect the urine sample in center of sample cup by releasing bulb. 4) The pipette content is transferred into a sample tube by squeezing bulb. 5) Steps 3 and 4 are repeated until the sample tube is filled to a ribbed line. 6) The sample tube is capped tightly and shaken vigorously for 20 seconds. 7) The sample tube with urine sample is placed in a sample bag and kept in refrigerator at 4° C. until ready to ship for deuterium measurement.

### Example 5: Sample Processing And Deuterium Level Measurement

Provided herein is an exemplary embodiment for processing a sample collected from a subject for deuterium level measurement. The sample is treated with a salt to precipitate the proteins present in the sample. The protein can be precipitated by adding 5 μg of salt, such as zinc sulfate monohydrate (ZnSO₄.H₂O) or other similar salts. The sample is centrifuged using an ultracentrifuge at 60,000 RPM, and the supernatant is removed to a new test tube and diluted with a volume of diluent with a known concentration of deuterium. The supernatant from the centrifuge was taken as is when the total volume of the supernatant was at least 600 μl. Alternatively, 300 μl of the supernatant was taken from the centrifuged tube and mixed with 700 μl of standard. Then, six 100 μl aliquots of each sample was measured for deuterium levels. Then, the average value was calculated for each subject. The final deuterium concentration was calculated using the dilution factor where appropriate.

After the sample is processed, the sample is measured for deuterium levels by isotope ratio mass spectrometry or water-based laser spectroscopy. The sample also can be measured using other analytical devices such as a mass spectrophotometer, gas spectrophotometer, or any other measuring tool that can quantitate hydrogen isotopes.

### Example 6: Analysis of Deuterium Level Measurement

Provided herein is an exemplary embodiment for analyzing the deuterium level measurements. The deuterium level measured from one or more of urine, saliva, blood, plasma, tear, or sweat sample provides the bodily fluid (BF) deuterium level. The deuterium level measured from breath condensate (BC) equals 50% BF deuterium level and 50% lungs and heart deuterium. Thus, BC deuterium level provides a marker for the deuterium level in tissues.

### Example 7: MRI Measurement of Deuterium Level

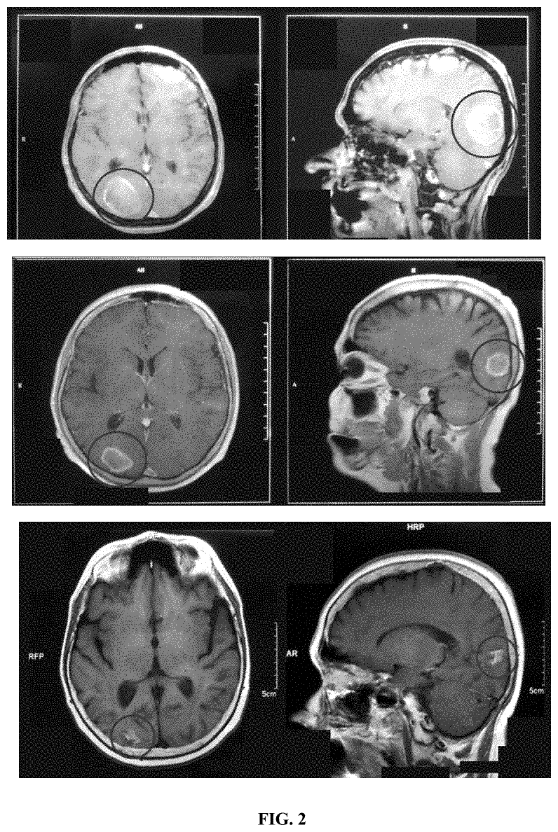

Provided herein is an exemplary embodiment for measuring the deuterium levels in tissue of interest in a human subject using MRI. FIG. 2 shows transverse and sagittal plane MM scan images of brains of a subject over a period of about three years. The images were taken by 1.5-T MM by a T1-weighted sequence with no contrast agents. Within one image, the whiter areas are indicative of a higher deuterium level than the darker areas. Regions with higher concentrations of deuterium, the whiter areas as marked by black circles, corresponded to cancerous tumors. The presence of cancer was also confirmed by other diagnostic means. A lower deuterium level was correlated with a decrease in a cancerous tumor, the decrease which was also demonstrated by other diagnostic means.

### Example 8: Deuterium Depletion Protocol

Provided herein is an exemplary embodiment for administration of a deuterium depletion protocol in a human subject. The subject is a 58 year old female who underwent a total mastectomy. The CT-scan of the subject showed a lung metastasis of 7.5×5.5×2.5 cm size before the mastectomy. The patient was administered and followed a deuterium depletion (DD) protocol about one month after the mastectomy in conjunction with chemotherapy. About six months after the DD protocol, the PET of the subject indicated no detectable metastasis. About four months afterward, the PET detected 3 different lymph nodes of pathologic size, which were treated with cytostatic medication and Herceptin. About two months later, the enlarged lymph nodes was not detectable by the control CT-scan. The subject has continued the DD protocol following the last CT-scan.

### Example 9: Deuterium Depletion Protocol

Provided herein is an exemplary embodiment for administration of a deuterium depletion protocol in a human subject. The subject is a 71 year old male patient. The subject had a PSA-value of 540 ng/mL and multiple bone metastases as verified by bone scintigraphy. Transurethal resection of the prostate (TURP) was performed after a biopsy. After the TURP procedure, a deuterium depletion (DD) protocol was administered and followed in conjunction with an anti-cancer medication protocol comprising Anandron, Suprefact depot, and Zometa. About three months later, the PSA value decreased to 2.79 ng/mL. About six months after the diagnosis, examinations verified a regression in the bone. A further improvement was seen in five months later. About two months later, the PSA-value was 1.8 ng/mL, and the subject was asymptomatic at that time.

### Example 10: Deuterium Depletion Protocol

Provided herein is an exemplary embodiment for administration of a deuterium depletion protocol in human subjects. The eight total human subjects were administered and followed a deuterium depletion (DD) protocol comprising either DD diet (4 subjects) or DD water (4 subjects). Breath condensate was collected using a breath condensate collector tube, and saliva was collected in a testing tube. The breath condensate and saliva of the subjects were measured for deuterium levels before and after the DD protocol using a liquid water isotope analyzer. The time length of REM cycle per 8 hours sleep in the subjects were measured before and after DD protocol. The control pause and fasting blood glucose levels of the subjects were measured before and after DD protocol. The results are presented in Table 2.

TABLE 2
Biomarkers in Subjects Before and After Deuterium Depletion Protocols

| | | | | | Blood | |
|---|---|---|---|---|---|---|
| | | | | | Glucose Levels | |
| | | Deuterium Levels | | | Control | Fasted |
| | | Breath | Saliva | REM | Pause | Glucose |
| | | [ppm] | [ppm] | Sleep/8 h | [mg/dL] | [mg/dL] |
| DD Diet | | | | | | |
| Subject 1 | Before | 148 | 155 | 30 min | 20 | 120 |
| | After | 139 | 143 | 145 min | 25 | 90 |
| Subject 2 | Before | 150 | 152 | 45 min | 35 | 110 |
| | After | 142 | 147 | 131 min | 37 | 85 |
| Subject 3 | Before | 148 | 152 | 61 min | 23 | 115 |
| | After | 141 | 146 | 82 min | 29 | 78 |
| Subject 4 | Before | 144 | 157 | 75 min | 27 | 98 |
| | After | 136 | 138 | 180 min | 30 | 81 |
| DD Water | | | | | | |
| Subject 5 | Before | 151 | 159 | 65 min | 27 | 138 |
| | After | 139 | 141 | 152 min | 31 | 91 |
| Subject 6 | Before | 150 | 152 | 170 min | 35 | 141 |
| | After | 129 | 133 | 169 min | 42 | 117 |
| Subject 7 | Before | 148 | 153 | 72 min | 10 | 210 |
| | After | 125 | 127 | 82 min | 19 | 146 |
| Subject 8 | Before | 145 | 149 | 150 min | 25 | 91 |
| | After | 120 | 120 | 160 min | 31 | 85 |

### Example 11: Deuterium Depletion Protocol For CCL Patients

Provided herein is an exemplary embodiment for administration of deuterium depletion protocols in human subjects affected by chronic lymphocytic leukemia (CLL).

Patients recently diagnosed with chronic lymphocytic leukemia (CLL) with asymptomatic disease (Binet A-B) are monitored without treatment, unless there is evidence of active disease. Patients with advanced disease (Binet C) and/or active disease are typically treated.

A majority of patients with early stage CLL (Binet A-B) at the time of diagnosis progress over time, requiring active treatment. Early initiation of chemotherapy in unselected early stage CLL patients has been associated with increased toxicity, but often has failed to show a survival benefit. Around 25% of Binet A patients are at a high risk of progression. These patients are monitored as there is currently no treatment available. It is a clear that a medical treatment option is highly needed as this high-risk patient population will increase. Therefore, previously untreated CLL patients that have asymptomatic disease (Binet A) but are at high risk of progression represent an appropriate patient population in which to test nutraceuticals such as deuterium depletion protocols (DD protocol).

By focusing on the target population of previously untreated CLL patients that have asymptomatic disease (Binet A) but are at high risk of progression a homogeneous patient population is selected. Around 80% of patients diagnosed with CLL are at stage Binet A. Of these patients ˜25% are at high risk of progression. This population represents around 20% of all CLL patients.

Although a retrospective study in CLL patients showed signs of efficacy with deuterium depletion therapy, it is currently unclear how efficacious DD protocol is in previously untreated CLL patients with asymptomatic disease (Binet A) who are at high risk of progression. Spontaneous regression are rare in patients with CLL (such remissions are seen in ˜1% of the cases). Therefore, any signs of efficacy seen in this study can be accounted to the DD protocol.

The aim of the treatment is to reduce the deuterium concentration in the patient's body by replacing the ordinary water intake with DD protocol. During the course of treatment, deuterium concentration is gradually decreased by the administration of DD protocol with decreasing levels of deuterium, resulting in a continuous decrease in deuterium concentration in the patient's body water pool. In the study, the deuterium levels in various biological fluids are measured before, during, and after the completion of treatment.

In vitro experiments with DDW showed that the cancer cells are sensitive to lowering deuterium concentration, but staying in media prepared with DDW for a longer period of time showed that the cells are able to adapt to the low deuterium concentration. The rationale for the gradual decrease of deuterium concentration in DDW is to cause a continuous decrease of deuterium concentration during the study.

A patient starts with a DD protocol inclusive of 85 ppm DDW for the first 8 weeks, followed by a DD protocol inclusive of 65 ppm DDW for the second 8 week and a DD protocol inclusive of 45 ppm DDW for the final 8 weeks. This type of dosing guarantees a continuously decreasing deuterium concentration in patients over 24 weeks. The gradually increasing dosing was used, because an in vitro experiment with DDW showed that the cancer cells are sensitive for lowering deuterium concentration and staying in media prepared with DDW for a longer period of time showed that the cells are able to adapt to the low deuterium concentration.

There is a strong correlation between the daily average deuterium concentration of water intake and the deuterium concentration in blood. Despite this strong correlation it is important to note that the final deuterium concentration of the plasma is modified by the water content of patient's food intake, the overall amount and the composition (ratio of carbohydrates, lipid and proteins) of the food. For a typical carbohydrate, the deuterium concentration is 150 ppm, but for butter and lipids only 115-125 ppm, which means the deuterium concentration of the metabolic water (150-300 ml/day) is influenced by carbon source oxidized in the cells. This shows that the low deuterium hydrogen available for ATP and metabolic water production in the mitochondria are provided via the four water recycling steps in the Krebs-Cycle.

In order to gain comparable data between patients a deuterium depletion unit (DdU) is introduced with the following formula: Water Dose (DdU)=150 (ppm)−deuterium concentration of DDW (ppm)×volume of DDW (L/day)/body weight (kg). In order to get optimal results the DdU may be equal or above. In addition, the actual deuterium level following the administration of a DD protocol was measured and the DdU was also calculated based on the ppm decrease in the tissue and/or biological fluid per volume of DDW consumed to standardize the actual biological activity of the specific DD protocol.

### Example 12: Deuterium Depletion Protocol

Provided herein is an exemplary embodiment for administration of a deuterium depletion protocol in human subjects. Chronic lymphocytic leukemia (CLL) patients and their physicians are in need of identifying low toxicity interventions with the potential to delay progressive disease, particularly for those patients whose molecular prognostic markers (such as ZAP-70 or IGHV mutation status,) to predict a higher risk of progressive disease. There are indications from clinical studies that various regiment of deuterium depleted water (DDW) can provide CLL patients with a new source of energy while increasing the potency of other treatments. One benefit of DDW is that it has little to no toxic effect on the patient as compared to many other CLL treatments.

A preliminary bioassay of CLL patients determines the level of deuterium in subject tissues. This is done with a non-invasive test of biofluids such as saliva and urine. Deuterium levels are calculated as part per million (ppm) in a subject's hydrogen atoms. Protocols for the intake of DDW vary based on the disease stage, patient condition, and on the physicians' recommended treatment. Nonetheless, it only requires a patient to drink DDW instead of regular water or other beverages. More bioassays are required during the course of treatment to monitor the decrease of deuterium level and adjust the lower level of deuterium in the DDW. Again, both testing and administration are non-toxic and non-invasive.

### Example 13: Deuterium Depletion Protocol

Provided herein is an exemplary embodiment for administration of a deuterium depletion protocol in human subjects. The effects of deuterium depleted water consumption was evaluated on 4 patients with brain metastases from lung cancer. The aim of the study was to investigate the impact of DDW consumption in addition to conventional forms of therapy on the survival of lung cancer patients with brain metastasis. A series of 4 case histories was retrospectively evaluated. The patients were diagnosed with brain metastasis deriving from a primary lung tumor and started consuming DDW at the time of or after the diagnosis of the brain metastasis, which was inoperable or the surgical intervention did not result in complete regression. The primary objective was survival. The daily water intake of the patients was replaced with DDW, which complemented the conventional forms of treatment. Patients were consuming DDW for at least 3 months. The treatment was continued with DDW of 10 to 15 to 20 ppm lower deuterium content every 1 to 2 months and thus a gradual decrease was maintained in the deuterium concentration in the patient's body. DDW consumption integrated in conventional treatments resulted in a survival time of 26.6, 54.6, 21.9, and 33.4 months in the 4 patients, respectively. The brain metastasis of 2 patients showed CR, whereas PR was detected in 1 patient, and the tumor growth was halted (no change) in 1 case.

### Example 14: Deuterium Depletion Protocol

Provided herein is an exemplary embodiment for administration of a deuterium depletion protocol in human subjects. The subjects were 74 metastatic breast cancer patients. The study evaluated the effect of deuterium depletion on survival. DDW therapy and conventional treatment were simultaneously administered. DDW consumption and simultaneous conventional treatment produced a response or halted progression in 74.3% of the 74 evaluated MBC patients (CR was detected in 16 cases (21.6%), 27 patients (36.5%) showed a PR while no change was observed in 12 patients (16.2%). Progressive disease was diagnosed in 19 women (25.7%). Median survival time (MST) from the start of DDW therapy was 33.6 months. The probability of surviving for periods of 1, 2, 5, and 8 years was 74.8%, 60.4%, 16.6%, and 12.5% respectively. Patients achieving CR or PR had a MST of up to 53 and 49 months respectively. The incidences of CR and PR were significantly higher (p=0.0028) in the subgroup of patients that received a higher dose of DDW and/or DDW of a lower deuterium content, i.e. the dose was more than 1.0 deuterium-depletion unit (DdU).

### Example 15: Deuterium Depletion Protocol

Provided herein is an exemplary embodiment for administration of a deuterium depletion protocol in human subjects. A human phase II clinical trial examined the effect of decreased deuterium containing water on patients with prostate tumor. The purpose of this study was to show the efficacy of deuterium depletion in prostate cancer patients. In the double blind, four-month-long, randomized Phase II clinical trial the daily water intake was replaced with DDW in 22 prostate cancer patients. Other 22 prostate cancer patients took normal water while both groups received the same forms of conventional treatment. In the retrospective study, 91 DDW-treated prostate cancer patients were evaluated and MST in the subgroups was calculated. The time course of changes in DDW dose and prostate specific antigen (PSA) is presented in two cases. In the trial seven patients in the treated group and one patient in the placebo group achieved PR (p=0.046). In the treated group, the net decrease in the prostate volume was three times higher (160.3 cm3 vs. 54.0 cm3; p=0.0019), urination complaints ceased at a higher rate (8 vs. 0 patients, p=0.0041), and the one-year survival was also longer (2 vs. 9 deaths; p=0.034). The 91 retrospectively evaluated patients achieved an MST of 11.02 years, despite the fact that 46 of them suffered from distant metastasis. In the two monitored patients, drop of PSA level correlated with the DDW intake. The deuterium depletion prolonged MST in patients with prostate cancer. The method proved to be safe thus its integration in the prostate cancer cure as an adjuvant or complementary therapy may be considered.

### Example 16: Deuterium Depletion Protocol

Provided herein is an exemplary embodiment for administration of a deuterium depletion protocol in human subjects. The subjects are pancreatic cancer patients. In this study the median age of the 32 DDW treated patients was 61.5 years. Median duration of the treatment with DDW was 6.64 months (85 ppm, 65 ppm and 45 ppm regimen DDW). The treatment period showed a high standard deviation (SD: 12.24 months), because 9 patients (28%) consumed DDW for an extended period, more than one year. Patients started the DDW treatment at different time points at their free consent after diagnosis, which was recorded. The median elapsed time between diagnosis and entering the trial was 1.16 months (SD: 4.06 months). When data sheets were closed for standard matrix diagnostics, data processing and abstracting, in order to meet study deadline objectives (June 2011), 17 patients were still alive and offered continued DDW treatment; therefore they are included in this report with their applicable (at least) mean survival time upon study termination. The median age of the 30 control natural deuterium-containing water consuming patients was 65.9 years (SD=9.05 years) (Female: n=16, Average age: 67 years, SD=9.24 years; Male: n=14, Average age: 64.3 years, SD=8.9). Histology in all cases confirmed pancreatic adenocarcinoma and all patients received treatment with standard gemcitabine (Gemzar 1000 mg/m² as an intravenous infusion) combined with 5 fluorouracil consistent with the EU gastrointestinal adenocarcinoma disease management protocol. Also, the nuclear membrane turnover and nucleic acid syntheses were investigated and determined by targeted [1,2-¹³C₂]-D-glucose fate associations. The results show that extra-mitochondrial NADPH synthesis pathways are targeted by DDW; particularly that of the direct glucose oxidizing arm of the pentose phosphate pathways, cytoplasmic fumarate hydratase and the serine oxidation glycine cleavage reactions to repair oncogenic NADPH producing glycolysis.

### Example 17: Deuterium Depletion Protocol

Provided herein is an exemplary embodiment for administration of a deuterium depletion protocol in human subjects. Fifty-three CLL patients underwent DDW treatment at least for 91 days, DDW was applied on its own or in addition to chemotherapy. Patients were treated and follow-up examinations were carried out at different Hungarian hospitals. The cumulative follow-up period from the diagnosis was 252.55 years, while the median follow-up was 3.98 years. Median follow-up from the start of DDW treatment was 14.07 months, the cumulative length of time was 116.3 years. Median duration of DDW treatment reached 11.7 months, the cumulative duration was 65.38 years. Patients started DDW treatment at different time points after the diagnosis, the median length of this period was 17.02 months (1.42 years). During the total cumulative period two deaths were registered. Patients starting the DDW treatment at an early stage of CLL showed improvement in their clinical status and blood counts. In advanced stages of CLL DDW treatment was initiated in addition to chemotherapy, and the results suggest, that deuterium depletion had an additive effect since patients achieved better results and had a longer symptom-free period. Twenty patients (37%) showed a response in terms of white blood cell counts, platelet counts and/or lymph node status. For 4 patients (7,5%) the clinical status was stable, while 7 patients (13%) showed progressive disease. For the rest of the patients no accurate follow-up data is available.

### Example 18: Deuterium Depletion Protocol

Provided herein is an exemplary embodiment for administration of a deuterium depletion protocol in human subjects. A toxicity and efficacy study in human subjects was performed. The results showed that patients belonging to the treated group did not suffer from any side effect of the agent. No white blood cell drop or any significant changes were found which may be the consequence of the DDW consumption after drinking 150-400 liters/patient.

Based on the above, treatment with DDW may be have efficacy and safety in oncological patient population. It is acknowledged that retrospective studies are not the gold standard for assessing efficacy and safety of DDW or drugs in general. Based on the available pre-clinical and clinical data, DDW appears to be safe and well tolerated.

### Example 19: In Vitro Study of Deuterium Depletion

The effect of deuterium concentration on the growth of L929 fibroblast cell lines in tissue culture was investigated. It was found that the start of cell proliferation in the deuterium depleted media was with a 6-8 hour lag period as compared to that of the culture grown in normal deuterium content media. The differences between cell growths were the most pronounced in the first 20 hours.

### Example 20: In Vitro Study of Deuterium Depletion

The Targeted 13C-Labeled Tracer Fate Associations study showed that deuterium depletion influences the metabolism of cancer cell lines. Deuterium incorporation from common water into DNA increases its fragility thus accelerating mutations, aging and cancer. Meanwhile deuterium depletion temporarily decelerates cell growth in vitro and induces tumor regression in vivo. The metabolic flux-modifying effects of deuterium depleted water (DDW: 100, 50 and 25 ppm deuterium) as compared to normal deuterium containing water (150 ppm) on [1,2-¹³C₂]-D-glucose metabolism in cultured pancreatic (MIA-PaCa), lung (H-441) and breast (MCF-7) ductal carcinoma cells were reported. After 72 hours of incubation with the tracer in DDW its uptake, lactate production, glycolysis, RNA ribose, glycogen, cholesterol and long chain fatty acid and TCA cycle glutamate syntheses, as well as ¹³CO₂ release using GC/MS was analyzed. DDW did not significantly alter glucose uptake, oxidation, glycogen, RNA ribose, lactate production or glucose oxidation in the TCA cycle in any of the cell lines. In MCF-7 cells cholesterol ¹³C labeling was increased by 4.3%, 50.2% and 66.5% during 100, 50 and 25 ppm water administration via HMG-CoA and mevalonate formation. In H441 and MIA cells, DDW induced a dose dependent, uniform and significant inhibition of fractional cholesterol and long-chain saturated fatty acid synthesis. Based on this data deuterium to hydrogen ratios regulate sterol and fatty acid precursor synthesis, which likely affects the rate of divisions and cellular proliferation via the regulation of reductive synthesis and new membrane formation.

### Example 21: In Vitro Study of Deuterium Depletion

In an in vitro toxicity test, the colony forming ability of cells was investigated. In addition, microscopic investigations were performed to assess any morphological changes due to deuterium depletion. The results showed that neither the decreased (35 ppm) nor the elevated (300 ppm) deuterium concentration influenced the colony forming ability of the Chinese Hamster Ovary (CHO) cells on toxic level (50% reduction in plating efficiency). The tumor cell viability was unaffected, suggesting that the depletion of deuterium in the culture media was not cytotoxic. The experiment showed little or no toxicological effects of DDW on cells in vitro.

### Example 22: In Vitro Study of Deuterium Depletion

An in-vivo toxicity test was performed to investigate the water and food uptake of the control and treated group, and to assess the increase in weight in the two groups in mice. The experimental animals were randomized into two dosage groups of DDW by gender, a D-15000 ppm dosage—control group (n=50) and a D-3000 ppm dosage of DDW—test group (n=51). Body weight, food and drink consumption, the sizes of the tumor were measured, symptoms were observed and histological samples were taken and evaluated regularly during the study. After three months the laboratory animals were killed in the control and treated groups and a comparative pathological study was carried out. In the organs of the abdominal and thoracic cavities no deformation was found due to the effect of the studied material. The animals, treated with D-3000 had considerably lower tumor growth intensity, smaller average tumor volume and much lower cumulative mortality rate than in the D-15000 dosage group. The experiment showed little or no toxicological effects of DDW on cells in vitro.

### Example 23: Water-Based Laser Spectroscopy

Provided herein is an exemplary embodiment for analyzing the deuterium level measurements using water-based laser spectroscopy. Various biological samples, including urine samples, can be analyzed by water-based laser spectroscopy. Two OA-ICOS laser absorption spectrometers (Los Gatos Research (LGR), Mountain View, Calif., USA) were used, one for simultaneous direct measurement of 2 H/1 H and 18O/16O (LWIA-V30d) and one for direct measurement of 17O/16O stable isotopes in liquid water. A urine sample was centrifuged and introduced into a OA-ICOS laser absorption spectrometer by a PAL HTC-xt autoinjector (CTC Analytics, Zwingen, Switzerland) equipped with a Hamilton 1.2 μl, zero dead-volume syringe and a heated (≈85° C.) injector block, where the water was evaporated for isotope analysis directly on the water vapor. In the OA-ICOS laser absorption spectrometer, a laser radiation is coupled to an optical cavity in an off-axis manner and is continuously measured similar to a standard laser absorption experiment. The cavity provides an extraordinarily long effective optical path length (typically 2-10 km), and the off axis configuration provides robustness, allowing for the accurate quantification of water isotopomers with very high precision.

### Example 24: MRI Luminescence Measurement

Provided herein is an exemplary embodiment for measuring the MRI tissue luminescence. The MRI tissue luminescent measurement uses a standard curve of DDW-filled sample containers. A standard curve of water with known DDW concentrations was prepared by mixing a certified standard containing 25 ppm water with a certified standard containing 155 ppm water. The resulting samples included the 155 ppm, 140 ppm, 125 ppm, 110 ppm, 90 ppm and 75 ppm. Three milliliters of each standard dilution was pipetted into 50×11mm plastic petri dishes using a glass pipette. The uncovered petri dishes containing the water was placed on the MRI bench with a subject and Mill performed accordingly at the same time as the subject. The resulting luminescence was plotted according to the samples and a consistent standard curve was generated.

### Example 25: Resting Metabolic Rate Measurement

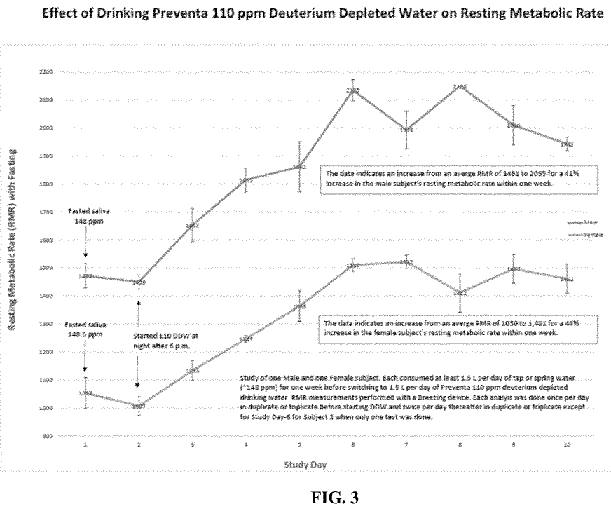

Provided herein is an exemplary embodiment for determining the efficacy of a deuterium depletion protocol by measuring the resting metabolic rate. The study had one male and one female subjects. Each consumed 1.5 L per day of tab or spring water (deuterium level of 148 ppm) for one week and then switched to consuming 1.5 L per day of 110 ppm DDW. The resting metabolic rate was measured with a breezing device every day during the study. The resting metabolic rate, or the basal amount of energy (Calories) required to power a body for a day, was increased in both patients by more than 40% in less than a week with the deuterium depletion protocol of consuming DDW as shown in FIG. 3.

### Example 26: Deuterium Depletion Protocol

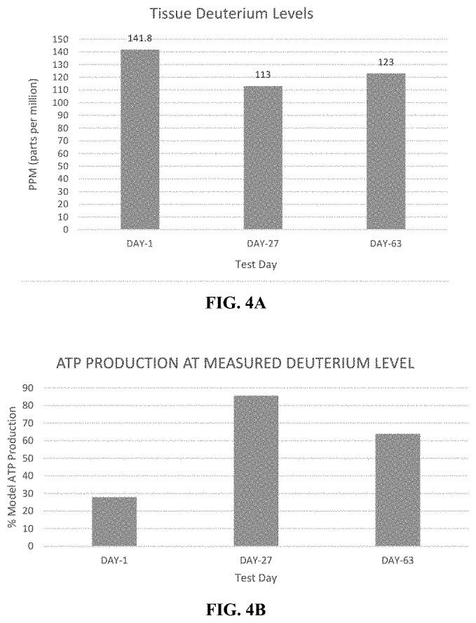

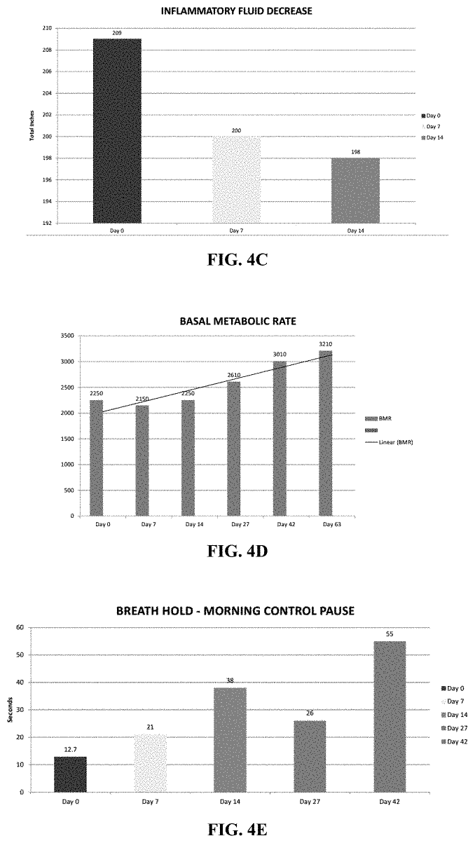

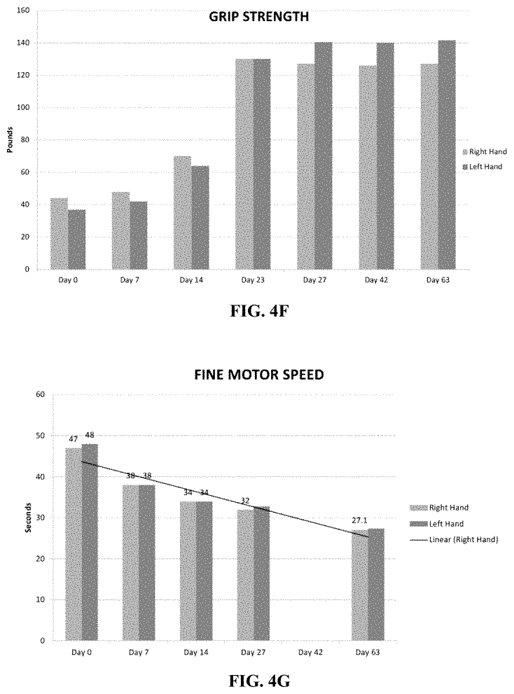

Provided herein is an exemplary embodiment of a deuterium depletion protocol administered to a human subject. The subject was diagnosed with Guillain-Barre syndrome, affecting the peripheral nervous system. The subject was tested once a week for strength, forced vital capacity, basal metabolic rate, GDV scan, various body metrics, and deuterium level of breath and saliva. The deuterium depletion treatment comprised deuterium depletion using water, food, and light with energy augmentation. The subject consumed 25 ppm deuterium depleted water and machine generated molecular water and a deuterium depleted diet with no additional supplements or medication. The subject underwent molecular hydrogen bath, home change of light and EMF, daily circadian rhythm entrainment, and UV and IR light. As shown in Table 3 and Table 4, improvements were observed over the course of the treatment in various metrics. FIG. 4A shows the tissue deuterium level and FIG. 4B shows ATP production measured at day 1, 27, and 63. FIG. 4C shows inflammatory fluid decrease at day 0, 7, and 14. FIG. 4D shows the basal metabolic rate at days 1, 7, 14, 27, 42, and 63. FIG. 4E shows the breath hold—morning control pause at day 0, 7, 14, 27, and 42. FIG. 4F shows the grip strength of right and left hands at days 0, 7, 14, 27, 42, and 63. FIG. 4G shows the fine motor speed of right and left hands at days 0, 7, 14, 27, and 63.

TABLE 3
Measurements at day 1 and day 7 of deuterium depletion treatment.

| | 1st day of encounter | 7 days on intervention | (Percentage Change) |
|---|---|---|---|
| | Nov. 25, 2017 | Dec. 2, 2017 | of first 14 days. |
| Tissue deuterium Level (ppm) | 141.8 | | |
| Tape measure of body | | | |
| Arms 11 cm up AC | R = bicep 35; forearm 30.5 | R = bicep 33.5; Forearm 30 | −2 cm; −3% decrease |
| 7 cm down AC | L = bicep 34.5; forearm 31 | L = bicep 34; forearm 29.5 | −2 cm; −3% decrease |
| Chest | nippleline = 103; Waist = 94 | nippleline 101.5; waist 91 | −4.5 cm = −2.2% decrease |
| Legs 17 cm patella | R = Thigh = 63; Calf = 38.5 | R = thigh 57; calf 37 | −7.5 cm = −7.3% decrease |
| 12 cm down patella | L = Thigh = 62; Calf = 39 | L = thigh 56.5; calf 37 | −7.5 cm = −7.4% decrease |
| Arms + Legs | 131 + 202.5 = 333.5 cm | 127 + 187.5 = 314.5 cm | −19 cm = −5.7%; −7.5 inches |
| Arms + Chest + legs total | 333.5 + 197 = 530.5 cm | 314.5 + 192.5 = 507 cm | −23.5 cm = −4.4%; −9.25 inches1 |
| Breath hold | 12.7 seconds | 21 seconds | 65% increase |
| Grip strength | R = 37/45/50 = 44 avg | R = 48/47/45 = 48 avg | 9% increase |
| | L = 40/34/39 = 37 avg. | L = 48/45/42 = | 14% increase |
| Basal metabolic rate | 2250 measured | 2350 | 4.4% increase |
| Weight | 206 | 202 | −4 lbs = −2% decrease |
| GDV scan | 51 Joules, 30 free energy | 49 J, 30 free energy | No change. |
| Visceral fat | 11% | 11% | No change. |
| Total fat bioimped. | 22.8 | 24.2 | increase 1.4% |
| Pulse ox and HR | 95% and 72 | 95% and 79 | No change. |
| Joint pain - subjective | range = 3-7/10 | range = 3-7/10 | No change. |
| Energy Level - subj. | 3/10 | 6/10 | 100% increase |
| Fine motor: alternate thumb | R = 47 seconds | R = 38 | 19% faster |
| | L = 48 seconds | L = 38 | 21% faster |

TABLE 4
Measurements at day 14, 27, 42, and 63 of deuterium depletion treatment.

| | 14 days intervetion | 27 days intervention | 42 days intervention | 63 days intarvention |
|---|---|---|---|---|
| | Dec. 9, 2017 | Dec. 22, 2017 | Jan. 6, 2018 | Jan. 27, 2018 |
| Tissue deuterium Level | | 113 | | 123 |
| (ppm) | | | | |
| Tape measure of body | | | | |
| Arms 11 cm up AC | R = bicep 33; forearm 29 | R = bicep 34.5; forearm 29.5 | R = bicep 33.5; forearm 29.5 | R = bicep 36; forearm 30 |
| 7 cm down AC | L = bicep 33.5; forearm 29.5 | L = bicep 33.5; forearm 30 | L = bicep 33.5; forearm 29.5 | L = bicep 35; forearm 30 |
| Chest | nippleline = 100; waist 91 | nippleline = 100.5; waist 91 | nippleline = 99; waist 93 | nippleline = 101; waist 88 |
| Legs 17 cm patella | R = thigh 57; calf 37 | R = thigh 57; calf 38.5 | R = thigh 59.5; calf 39 | R = thigh 61; calf 38 |
| 12 cm down patella | L = thigh 57; calf 37 | L = thigh 60; calf 38.5 | L = thigh 59.5; calf 38.5 | L = thigh 60.5; calf 38 |
| Arms + Legs | 125.5 + 188 = 313.5; | 127.5 + 194 = 321.5 | 126 + 196.5 = 322.5 | 131 + 197. = 328.5 |
| Arms + Chest + legs total | 313.5 + 191 = 504.5 | 321.5 + 191.5 = 513 | 322.5 + 192 = 514.5 | 328.5 + 189 = 517.5 |
| Breath hold | 38 seconds | 26 seconds | 55.75 seconds | |
| Grip strength | R = 80/63/67 = 70 | R = 128/126 = 127 avg | R = 124/128 = 126 avg | 128/126 = 127 |
| | L = 65/65/62 = 64 | L = 140/141 = 140.5 avg | L = 140/140 = 140 avg | 145/138 = 141.5 |
| Basal metabolic rate | 2250 | 2610 | 3010 | 3620 |
| Weight | 205 | 207.8 | 208 | 207.2 |
| GDV scan | 40 J | 43 J | 41 J | 45 |
| Visceral fat | 11 | 11 | 11 | 11 |
| Total fat bioimped. | 23.7 | 25.1 | 23.3 | 26.8 |
| Pulse ox and HR | 97% | 87 | 97% | 96 |
| Joint pain - subjective | Mar. 7, 2010 | | | |
| Energy Level - subj. | 10-Jun | | | |
| Fine motor: alternate thumb | 34.6 seconds | 32 | | 27.1 |
| | 34.6 seconds | 32.8 | | 27.4 |

### Exemplary Embodiments

Among the exemplary embodiments are: 1. Methods of measuring a deuterium level to determine a rate of deuterium depletion in a subject comprising: a) obtaining a first biological fluid from the subject; b) measuring an initial level of deuterium in the first biological fluid; c) providing a deuterium-depletion protocol to the subject; d) obtaining a subsequent biological fluid from the subject; and e) measuring a subsequent level of deuterium in the subsequent biological fluid, wherein the initial level and the subsequent level are used to determine a rate of deuterium depletion in the subject. 2. The method of embodiment 1, wherein the deuterium-depletion protocol comprises providing a deuterium-depleted water to the subject. 3. The method of embodiment 1, wherein the deuterium-depletion protocol comprises providing a deuterium-depleted nutritional diet to the subject. 4. The method of embodiment 1, wherein the deuterium-depletion protocol comprises providing a ketogenic nutritional protocol to the subject. 5.The method of embodiment 1, wherein the biological fluid comprises blood, serum, plasma, urine, saliva, tear, sweat, or breath condensate, stool, or spinal fluid. 6. The method of embodiment 1, wherein the biological fluid comprises blood, serum, plasma, urine, saliva, or breath condensate. 7. The method of embodiment 1, wherein the biological fluid is obtained from exhaled breath of the subject. 8. The method of embodiment 1, wherein steps d) and e) are repeated over a pre-determined period of time. 9. The method of embodiment 8, wherein the pre-determined period of time is at least one week. 10. The method of embodiment 2, wherein the deuterium depleted water comprises about 65 ppm to about 135 ppm deuterium. 11. The method of embodiment 10, wherein the deuterium depleted water comprises about 65 ppm to about 125 ppm deuterium. 12. The method of embodiment 3, wherein the deuterium-depleted nutritional diet comprises a natural plant derived diet, an animal fat derived diet, or a combination thereof. 13. The method of embodiment 3, wherein the deuterium-depleted nutritional diet comprises a vegan diet. 14. The method of embodiment 3, wherein the deuterium-depleted nutritional diet comprises plant or animal grown in low deuterium environment. 15. The method of embodiment 3, wherein the deuterium-depleted nutritional diet comprises a diet having a predetermined deuterium content. 16. The method of embodiment 1, wherein the deuterium-depletion protocol comprises an anti-cancer agent. 17. The method of embodiment 1, wherein measuring of steps b) and e) comprises measuring deuterium level using isotope ratio mass spectrometry. 18. The method of embodiment 1, wherein measuring of steps b) and e) comprises measuring deuterium level using water-based laser spectroscopy. 19. The method of embodiment 1, wherein measuring of steps b) and e) comprises measuring a deuterium-proton ratio of the biological fluid. 20. The method of embodiment 1, wherein the subject is a human. 21. The method of embodiment 1, wherein the subject is an animal. 22. The method of embodiment 1, wherein the subject is a plant. 23. The method of embodiment 1, wherein the subject is a food item.

24. Methods of measuring a deuterium level in a subject comprising: a) measuring an initial deuterium level in a tissue of interest the subject; b) providing a deuterium-depletion protocol to the subject; c) measuring a subsequent deuterium level in the tissue of interest in the subject, wherein the initial deuterium level and the subsequent deuterium level are used to determine a rate of deuterium depletion level in the subject, wherein the measuring of steps a) and c) comprises measuring deuterium level in the tissue of interest using MM. 25. The method of embodiment 24, wherein the deuterium-depletion protocol comprises providing a deuterium-depleted water to the subject. 26. The method of embodiment 24, wherein the deuterium-depletion protocol providing a ketogenic nutritional protocol to the subject. 27. The method of embodiment 24, wherein the measuring deuterium levels in tissues using MM comprises measuring Mill tissue luminescence. 28. The method of embodiment 24, wherein the measuring comprises proton (1H) nuclear MM of the tissue of interest in the subject. 29. The method of embodiment 24, wherein the measuring comprises determining a deuterium-proton ratio in the tissue of interest in the subject. 30. The method of embodiment 29, wherein a decrease in the deuterium-proton ratio indicates deuterium depletion. 31. The method of embodiment 24, wherein the tissue of interest comprises at least one of skin, muscle, and adipose tissue. 32. The method of embodiment 24, wherein the subject is a human. 33. The method of embodiment 24, wherein the subject is an animal. 34. The method of embodiment 24, wherein the subject is a plant. 35. The method of embodiment 24, wherein the subject is a food item.

36. The method of embodiment 1, wherein the measuring further comprises measuring of a tissue of interest in the individual using MRI. 37. The method of embodiment 1, wherein the biological fluid is breath condensate and blood. 38. The method of embodiment 1, wherein the measuring comprises determining a weighted average of deuterium values from breath condensate and blood. 39. The method of embodiment 1, wherein the deuterium-depletion protocol comprises providing a breathing protocol in an environment comprising water vapor having a deuterium level of no more than 135 ppm. 40. The method of embodiment 1, wherein the deuterium-depletion protocol comprises exposure to red or near infra-red light for a pre-determined length of time. 41. The method of embodiment 1, wherein the deuterium-depletion protocol comprises providing a wash protocol with a wash composition having a deuterium level of no more than 135 ppm. 42. The method of embodiment 1, wherein the deuterium-depletion protocol comprises providing a topical composition having a deuterium level of no more than 135 ppm.

43. The method of embodiment 19, wherein the deuterium-proton ratio of the biological fluid below 1:5000 is indicative of deuterium depletion. 44. The method of embodiment 24, wherein the measuring comprises using a 1.5 Tesla or lower magnetic field MM. 45. The method of embodiment 24, wherein the measuring comprises using a T1 sequence. 46. The method of embodiment 24, wherein the measuring comprises using a T1 sequence without contrast. 47. The method of embodiment 24, wherein the measuring comprises measuring proton tunneling, wherein the proton tunneling measures free proton movements. 48. The method of embodiment 29, wherein the deuterium-proton ratio in the tissue of interest in the subject below 1:5000 is indicative of deuterium depletion. 49. The method of embodiment 24, wherein the measuring further comprises measuring a biological fluid obtained from the subject.

50. Methods of treating a metabolic disease in a subject comprising: a) measuring an initial deuterium level in a first biological fluid from the subject; b) providing a deuterium-depletion protocol to the subject; c) measuring a subsequent deuterium level in a subsequent biological fluid, wherein the initial deuterium level and the subsequent deuterium level are used to determine a status of the metabolic disease in the subject. 51. The method of embodiment 50, wherein the deuterium-depletion protocol comprises a diet comprising food having deuterium level of no more than 135 ppm. 52. The method of embodiment 50, wherein the deuterium-depletion protocol comprises exposure to red and near-infra-red light. 53. The method of embodiment 50, wherein the deuterium-depletion protocol comprises a breathing method to enhance deuterium depletion. 54. The method of embodiment 50, wherein the deuterium-depletion protocol comprises a method to increase breath fractionation. 55. The method of embodiment 50, wherein the deuterium-depletion protocol comprises breathing in an environment filled with vapors having a deuterium level of no more than 135 ppm. 56. The method of embodiment 50, wherein the deuterium-depletion protocol comprises a sleeping method to enhance deuterium depletion. 57. The method of embodiment 50, wherein the deuterium-depletion protocol comprises cold and hot thermotherapy or thermogenesis. 58. The method of embodiment 50, wherein the deuterium-depletion protocol comprises sound therapy. 59. The method of embodiment 50, wherein the deuterium-depletion protocol comprises at least one of acupuncture, chiropractic manipulation, and massage therapy. 60. The method of embodiment 50, wherein the deuterium-depletion protocol comprises an ozone and hyperbaric oxygen therapy. 61. The method of embodiment 50, wherein the deuterium-depletion protocol comprises injecting a composition having a deuterium level of no more than 135 ppm into a tissue or a blood vessel of the subject. 62. The method of embodiment 50, wherein the deuterium-depletion protocol comprises applying a topical composition having a deuterium level of no more than 135 ppm on a skin of the subject. 63. The method of embodiment 50, wherein the deuterium-depletion protocol comprises administering washes and cleanses with a composition having a deuterium level of no more than 135 ppm to at least one of ear, eyes, nose, colon, vagina or penis. 64. The method of embodiment 50, wherein the deuterium-depletion protocol comprises cryotherapy. 65. The method of embodiment 50, wherein the metabolic disease comprises at least one of diabetes, acid-base imbalance, metabolic brain diseases, calcium metabolic disorders, DNA repair-deficiency disorders, glucose metabolism disorders, hyperlactemia, iron metabolism disorders, lipid metabolism disorders, malabsorption syndromes, metabolic syndrome X, inborn error of metabolism, enzyme deficiencies, mitochondrial diseases, phosphorus metabolism disorders, porphyrias, proteostasis deficiencies, metabolic skin diseases, wasting syndrome, and water-electrolyte imbalance. 66. The method of embodiment 65, wherein the metabolic disease is diabetes. 67. The method of embodiment 50, wherein the metabolic disease is cancer. 68. The method of embodiment 67, wherein the cancer comprises a tumor of at least one of breast, heart, lung, small intestine, colon, spleen, kidney, bladder, head, neck, ovarian, prostate, brain, pancreatic, skin, bone, bone marrow, blood, thymus, uterine, testicular, liver, or combinations thereof. 69. The method of embodiment 50, wherein the method results in an increase in ATP production. 70. The method of embodiment 50, wherein the method results in an increase in metabolic water production. 71. The method of embodiment 50, wherein the method results in improved cellular function. 72. The method of embodiment 50, wherein the method mitigates the progression of the metabolic disease. 73. The method of embodiment 50, wherein the method prevents growth of or reduces a tumor. 74. The method of embodiment 50, wherein the method enhances creation of a biologically active molecule having a favorable three-dimensional structure. 75. The method of embodiment 73, wherein the biologically active molecule comprises cholesterol, estrogen, or DNA. 76. The method of embodiment 50, wherein the measuring comprises using water-based laser spectroscopy, isotope ratio mass spectrometry, or proton (1H) nuclear MRI. 77. The method of embodiment 51, wherein the diet comprising food having deuterium level of no more than 135 ppm comprises at least one of vegan, vegetarian, ketogenic, or low carbohydrate diet. 78. The method of embodiment 53, wherein the breathing depletes deuterium during sleep. 79. The method of embodiment 53, wherein the breathing depletes deuterium by lymphatic drainage. 80. The method of embodiment 55, wherein the environment filled with vapors having a deuterium level of no more than 135 ppm is prepared by dehumidifying the environment, humidifying with a deuterium-depleted water, or a combination thereof. 81. The method of embodiment 56, wherein the sleeping method comprises use of blue-light blocking glasses or sound therapy or a combination thereof. 82. The method of embodiment 56, wherein the sleeping method increases a melatonin level in the subject. 83. The method of embodiment 57, wherein the cold and hot thermotherapy or thermogenesis burns adipose tissue of the subject. 84. The method of embodiment 57, wherein the cold and hot thermotherapy or thermogenesis enhances sleep length or REM cycle length in the subject. 85. The method of embodiment 59, wherein the at least one of acupuncture, chiropractic manipulation, and massage therapy improved function of lymphatics or a digestive system of the subject. 86. The method of embodiment 60, wherein the ozone and hyperbaric oxygen therapy provides an oxygen level of greater than 21%. 87. The method of embodiment 62, wherein applying the topical composition on the skin decreases a deuterium level of the skin. 88. The method of embodiment 62, wherein applying the topical composition on the skin decreases a deuterium level of skin microbiome. 89. The method of embodiment 63, wherein administering washes and cleanses decreases a deuterium level of a skin of the subject. 90. The method of embodiment 63, wherein administering washes and cleanses decreases a deuterium level of skin microbiome. 91. The method of embodiment 50, wherein the deuterium-depleting protocol is used in combination with an anti-cancer agent. 92. The method of embodiment 50, wherein the deuterium-depleting protocol is used in combination with a conventional therapy for the metabolic disease. 93. The method of embodiment 50, wherein the method prevents weight loss. 94. The method of embodiment 50, wherein the method prevents in weight gain.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of measuring a deuterium level to determine a rate of deuterium depletion in a subject comprising:
a) obtaining a first biological fluid from the subject;
b) measuring an initial level of deuterium in the first biological fluid;
c) providing a deuterium-depletion protocol to the subject;
d) obtaining a subsequent biological fluid from the subject; and
e) measuring a subsequent level of deuterium in the subsequent biological fluid, wherein the initial level and the subsequent level are used to determine a rate of deuterium depletion in the subject.

2. The method of claim 1, wherein the deuterium-depletion protocol comprises providing a deuterium-depleted water to the subject.

3. The method of claim 1, wherein the deuterium-depletion protocol comprises providing a deuterium-depleted nutritional diet to the subject.

4. The method of claim 1, wherein the biological fluid comprises blood, serum, plasma, urine, saliva, tear, sweat, or breath condensate, stool, or spinal fluid.

5. The method of claim 2, wherein the deuterium depleted water comprises about 65 ppm to about 135 ppm deuterium.

6. The method of claim 3, wherein the deuterium-depleted nutritional diet comprises a natural plant derived diet, an animal fat derived diet, or a combination thereof.

7. The method of claim 1, wherein the deuterium-depletion protocol comprises an anti-cancer agent.

8. The method of claim 1, wherein measuring of steps b) and e) comprises measuring deuterium level using isotope ratio mass spectrometry.

9. The method of claim 1, wherein measuring of steps b) and e) comprises measuring deuterium level using water-based laser spectroscopy.

10. The method of claim 1, wherein measuring of steps b) and e) comprises measuring a deuterium-proton ratio of the biological fluid.

11. A method of measuring a deuterium level in a subject comprising:
a) measuring an initial deuterium level in a tissue of interest the subject;
b) providing a deuterium-depletion protocol to the subject;
c) measuring a subsequent deuterium level in the tissue of interest in the subject, wherein the initial deuterium level and the subsequent deuterium level are used to determine a rate of deuterium depletion level in the subject,
wherein the measuring of steps a) and c) comprises measuring deuterium level in the tissue of interest using MRI.

12. The method of claim 11, wherein the deuterium-depletion protocol comprises providing a deuterium-depleted water to the subject.

13. The method of claim 11, wherein the measuring deuterium levels in tissues using MM comprises measuring MRI tissue luminescence.

14. The method of claim 11, wherein the measuring comprises proton (¹H) nuclear MM of the tissue of interest in the subject.

15. The method of claim 11, wherein the tissue of interest comprises at least one of skin, muscle, and adipose tissue.

16. The method of claim 1, wherein the measuring comprises determining a weighted average of deuterium values from breath condensate and blood.

17. A method of treating a metabolic disease in a subject comprising:
a) measuring an initial deuterium level in a first biological fluid from the subject;
b) providing a deuterium-depletion protocol to the subject;
c) measuring a subsequent deuterium level in a subsequent biological fluid, wherein the initial deuterium level and the subsequent deuterium level are used to determine a status of the metabolic disease in the subject.

18. The method of claim 17, wherein the deuterium-depletion protocol comprises a diet comprising food having deuterium level of no more than 135 ppm.

19. The method of claim 17, wherein the deuterium-depletion protocol comprises exposure to red and near-infra-red light.

20. The method of claim 17, wherein the measuring comprises using water-based laser spectroscopy, isotope ratio mass spectrometry, or proton (¹H) nuclear MRI.
